SEQUENCE LISTING (25 sequences)

SEQ ID NO: 1 - DNA, 2828 bp, Homo sapiens
  CDS (411)..(1448)
agcagccggcacggggacagccggccgcacaacggatctgcaggcgcggagcaaaatgcacccgccgcgccgcgcggtcctgcagccccgccacggccccgcggcccgcacccccccggggcgacagtgagcctctcccgccaccaccgggggccgagcggagggctctcgggtgggagagcgggaccagatctcgacagctgttcatttccaggaagccaccgcagccagagcgaaaggggaccttctgccaccagcggggcatcagccagcggcgcgcatggatttatgaagacactcatgcaagaagtgggcaggacttggacaaacttttccaccggctccgcgtccgccgctccccgcgcctcgtctcctttcccctcctctcccggcggccgccgctgcccgcgatggtggccgcgctgctgggcggcggcggcgaggcccgcggggggacagtgccgggcgcctggctgtgcctgatggcgctgctgcagctgctgggctcggcgccgcggggatcggggctggcgcacggccgccgcctcatctgctggcaggcgctgctgcagtgccagggggagccggagtgcagctacgcctacaaccagtacgccgaggcgtgcgcgccggtgctggcgcagcacggcgggggcgacgcgcccggggccgccgccgccgctttcccggcctcggccgcctctttctcgtcgcgctggcgctgcccgagtcactgcatctcggccctcattcagctcaaccacacgcgccgcgggcccgccctggaggactgtgactgcgcgcaggacgagaactgcaagtccaccaagcgcgccattgagccgtgcctgccccggacgagcggcggcggcgcgggcggccccggcgcgggcggggtcatgggctgcaccgaggcccggcggcgctgcgaccgcgacagccgctgcaacctggcgctgagccgctacctgacctactgcggcaaagtcttcaacgggctgcgctgcacggacgaatgccgcaccgtcattgaggacatgctggctatgcccaaggtggcgctgctcaacgactgcgtgtgcgacggcctcgagcggcccatctgcgagtcggtcaaggagaacatggcccgcctgtgcttcggcgccgagctgggcaacggccccggcagcagcggctcggacgggggcctggacgactactacgatgaggactacgatgacgagcagcgcaccgggggcgcgggtggtgagcagccgctggacgacgacgacggcgtcccgcacccaccgcgcccgggcagcggcgctgctgcatcgggcggccgcggggacctgccctatgggcctgggcgcaggagcagcggcggcggcggccgcttggcgccccggggcgcctggaccccactcgcctccatcttgctgctgctgcttgggccgctcttttagccctcgcgccccccgccgttggctgcgggagagcccgcgtcccactcccgtgctcgcctcgaccccgcgccgggcacctgtggcttgggacagatagaagggatggttggggatacttcccaaaactttttccaagtcaacttggtgtagccggttccccggccacgactctgggcacttcccctgaagctcctctccggagcttgacttcttggacctcctcccccgccccaattccaagctccagaaactcccaactcgtctgccgtccagaaagctagctgcagtgttcaggacgtccgggaggaagcaagcatgtgggggacagaacagtagtcctggactcgaaagggaaggtgctgaccagtggggccttagcaatttgaagggttgggaaggaggaattatatttgcaaaggggctgtctattagcatatttcctttgagggggcaaaaaaaagtgccagtatcgacttttacagattgtggccagtgaggatattataatcctatgtaaacagaaaagtcccacttaccgattcattctttcactgtttgtatctgcgcccagaattctcagtgacgtgggggtgagggtgggtggcgattgccttagagggaacccctaaattggttttggataagtttgagcccttgaccttaatttcattgctaccactctgatctcttagcacatttcttaggattaagggtccaaaaatgctgatctaaggggttgccatggtgttgaacaatgcaactttttatttaaaaaagctctgcactgccatgtatgaaagtctctttatgatgtttgtttttttgtcatttttgttctttacatcaagaaattttatgtttaaatatgcggagaatgtatattgcctctgctcctatcagggttgctaaaccctggtacatcgtatataaaatgtattaaaactggggtttgttaccagttgctgtactttgtatatagaatttttataaattgtatgcttcagaaataatttatttttaaaaagaaattaaaagttttaaactcacatccatattacacctttcccccctgaaatgtatagaatccatttgtcatcaggaatcaaaacccacagtccattgtgaagtgtgctatatttagaacagtcttaaaatgtacagtgtattttatagaattgaagttaacattcttattttcaagagaatttatggacgttgtagaaatgtacaaatgcatttccaaactgccttaaacgttgtatttttatagacatgtttttttaaaaatcctaagtttttaaataactatggatttgtgtattttttttggttatttgttttattaaaacatgtacatcagtaaagagttttaaacaatga

SEQ ID NO: 2 - protein, 345 aa, Homo sapiens
MVAALLGGGGEARGGTVPGAWLCLMALLQLLGSAPRGSGLAHGRRLICWQALLQCQGEPECSYAYNQYAEACAPVLAQHGGGDAPGAAAAAFPASAASFSSRWRCPSHCISALIQLNHTRRGPALEDCDCAQDENCKSTKRAIEPCLPRTSGGGAGGPGAGGVMGCTEARRRCDRDSRCNLALSRYLTYCGKVFNGLRCTDECRTVIEDMLAMPKVALLNDCVCDGLERPICESVKENMARLCFGAELGNGPGSSGSDGGLDDYYDEDYDDEQRTGGAGGEQPLDDDDGVPHPPRPGSGAAASGGRGDLPYGPGRRSSGGGGRLAPRGAWTPLASILLLLLGPLF

SEQ ID NO: 3 - DNA, 5855 bp, Homo sapiens
  CDS (319)..(1461)
agttgcctgcgcgccctcgccggaccggcggctccctagttgcgccccgaccaggccctgcccttgctgccggctcgcgcgcgtccgcgccccctccattcctgggcgcatcccagctctgccccaactcgggagtccaggcccgggcgccagtgcccgcttcagctccggttcactgcgcccgccggacgcgcgccggaggactccgcagccctgctcctgaccgtccccccaggcttaacccggtcgctccgctcggattcctcggctgcgctcgctcgggtggcgacttcctccccgcgccccctccccctcgccatgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagatttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattattccaaaagttttttgagtcttgagcttgggctgtggccctgctgtgatcataccttgagcacgacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttactgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctgcacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgaataagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatatcttgtacttttctattaaaatcattttatgaaatccaaaaaaaaaaaaaaaaaa

SEQ ID NO: 4 - protein, 380 aa, Homo sapiens
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 5 - DNA, 1598 bp, Homo sapiens
  CDS (85)..(915)
gcgcacgtcccggattcctcccacgagggggcgggctgcggccaaatctcccgccaggtcagcggccgggcgctgattggccccatggcggcggggccggctcgtgattggccagcacgccgtggtttaaagcggtcggcgcgggaaccaggggcttactgcgggacggccttggagagtactcgggttcgtgaacttcccggaggcgcaatgagctgcattaacctgcccactgtgctgcctggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcactgcccgcctgcctgctccgagacgtggcccaggaggccctgggcgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatgcagccctgccaactgagggacctgcgagggccgcccgctcccttcctgccactgccgcctactggacgctgccctgcatgctgcccagccactccaggaggaagtcgggaggcgtggagggtgaccacaccttggccttctgggaactctcctttgtgtggctgccccacctgccgcatgctccctcctctcctacccactggtctgcttaaagcttccctctcagctgctgggacgatcgcccaggctggagctggccccgcttggtggcctgggatctggcacactccctctccttggggtgagggacagagccccacgctgttgacatcagcctgcttcttcccctctgcggctttcactgctgagtttctgttctccctgggaagcctgtgccagcacctttgagccttggcccacactgaggcttaggcctctctgcctgggatgggctcccaccctcccctgaggatggcctggattcacgccctcttgtttccttttgggctcaaagcccttcctacctctggtgatggtttccacaggaacaacagcatctttcaccaagatgggtggcaccaaccttgctgggacttggatcccaggggcttatctcttcaagtgtggagagggcagggtccacgcctctgctgtagcttatgaaattaactaattgaaaattcactggttggtggaaaa

SEQ ID NO: 6 - protein, 276 aa, Homo sapiens
MAAGPARDWPARRGLKRSAREPGAYCGTALESTRVRELPGGAMSCINLPTVLPGSPSKTRGQIQVILGPMFSGKSTELMRRVRRFQIAQYKCLVIKYAKDTRYSSSFCTHDRNTMEALPACLLRDVAQEALGVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGKPGEAVAARKLFAPQQILQCSPAN

SEQ ID NO: 7 - DNA, 20 bp, Artificial Sequence
  forward primer for qRT PCR assay of RPL13a
gtacgctgtgaaggcatcaa

SEQ ID NO: 8 - DNA, 19 bp, Artificial Sequence
  reverse primer for qRT PCR assay of RPL13a
gttggtgttcatccgcttg

SEQ ID NO: 9 - DNA, 45 bp, Artificial Sequence
  Illumina probe
ggcgattgccttagagggaacccctaaattggttttggataagtt

SEQ ID NO: 10 - DNA, 47 bp, Artificial Sequence
  Illumina probe
tgggacagatagaagggatggttggggatacttcccaaaactttttc

SEQ ID NO: 11 - DNA, 42 bp, Artificial Sequence
  Illumina probe
gtggagagggcagggtccacgcctctgctgtacttatgaaat

SEQ ID NO: 12 - DNA, 44 bp, Artificial Sequence
  Illumina probe
ctggtgatggtttccacaggaacaacagcatctttcaccaagat

SEQ ID NO: 13 - DNA, 43 bp, Artificial Sequence
  Illumina probe
agttgatgagacgcgtccgtcgcttccagattgctcagtacaa

SEQ ID NO: 14 - DNA, 44 bp, Artificial Sequence
  Illumina probe
cactgggtcccctttggttgtaggacaggaaatgaaacatagga

SEQ ID NO: 15 - DNA, 45 bp, Artificial Sequence
  Illumina probe
ccatatttttctccttcgcccaggttgtaattgaagccaattctt

SEQ ID NO: 16 - DNA, 44 bp, Artificial Sequence
  Illumina probe
ggaggagaagcgcagtcaatcaacagtaaacttaagagaccccc

SEQ ID NO: 17 - DNA, 47 bp, Artificial Sequence; identical to SEQ ID NO: 10
  Illumina probe

SEQ ID NO: 18 - DNA, 23 bp, Artificial Sequence
  WNT5A primer
gtgcaatgtcttccaagttcttc

SEQ ID NO: 19 - DNA, 22 bp, Artificial Sequence
  WNT5A primer
ggcacagtttcttctgtccttg

SEQ ID NO: 20 - DNA, 22 bp, Artificial Sequence
  WNT5A primer
ggctggaagtgcaatgtcttcc

SEQ ID NO: 21 - DNA, 21 bp, Artificial Sequence
  WNT5A primer
gcctgtcttcgcgccttctcc

SEQ ID NO: 22 - DNA, 18 bp, Artificial Sequence
  TK1 primer
cgccgggaagaccgtaat

SEQ ID NO: 23 - DNA, 18 bp, Artificial Sequence
  TK1 primer
tcaggatggccccaaatg

SEQ ID NO: 24 - DNA, 22 bp, Artificial Sequence
  GAS1 primer
aatacattgctcaccaggaacc

SEQ ID NO: 25 - DNA, 22 bp, Artificial Sequence
  GAS1 primer
gtttaaggcagtttggaaatgc

(19) United States
(12) Patent Application Publication
(10) Pub. No.: US 2010/0196902 A1
(43) Pub. Date: Aug. 5, 2010

(54) PROSTATE CANCER BIOMARKERS

(76) Inventors: Gary Pestano, Oro Valley, AZ (US); Ubaradka G. Satilayanarayana, Tucson, AZ (US); Janice Riley, Tucson, AZ (US); Ray B. Nagle, Tucson, AZ (US)

Correspondence Address: KLARQUIST SPARKMAN, LLP, 121 S.W. SALMON STREET, SUITE 1600, PORTLAND, OR, 97204, US

(21) Appl. No.: 12/677,324

(22) PCT Filed: Sep. 15, 2008
(86) PCT No.: PCT/US08/76407

§ 371 Date: Mar. 10, 2010

Related U.S. Application Data
(60) Provisional application No. 61/054,925, filed on May 21, 2008.
(60) Provisional application No. 60/972,694, filed on Sep. 14, 2007.

(51) Int. Cl. C12Q 1/68; C40B 40/06

(52) U.S. Cl. 435/6; 506/16

(57) ABSTRACT

Disclosed are biomarkers, at least, useful for the diagnosis and/or prognosis of cancer and for making treatment decisions in cancer, for example prostate cancer.

### CROSS REFERENCE TO RELATED APPLICATIONS

This application claims the benefit of U.S. Provisional Application Nos. 60/972,694 filed Sep. 14, 2007 and 61/054,925 filed May 21, 2008, both herein incorporated by reference.

### ACKNOWLEDGMENT OF GOVERNMENT SUPPORT

This invention was made with United States government support pursuant to grant no. PO1 CA 56666 from the National Institutes of Health; the United States government has certain rights in the invention.

### FIELD

Disclosed herein are biomarkers, at least, useful for the diagnosis and/or prognosis of cancer and for making treatment decisions in cancer, for example prostate cancer.

### BACKGROUND

Oncologists have a number of treatment options available to them, including different combinations of chemotherapeutic drugs that are characterized as “standard of care,” and a number of drugs that do not carry a label claim for particular cancer, but for which there is evidence of efficacy in that cancer. The best chance for a good treatment outcome requires that patients promptly receive optimal available cancer treatment(s) and that such treatment(s) be initiated as quickly as possible following diagnosis. On the other hand, some cancer treatments have significant adverse effects on quality of life; thus, it is equally important that cancer patients do not unnecessarily receive potentially harmful and/or ineffective treatment(s).

Prostate cancer provides a good case in point. In 2008, it is estimated that prostate cancer alone will account for 25% of all cancers in men and will account for 10% of all cancer deaths in men (Jemal et al., CA Cancer J. Clin. 58:71-96, 2008). Prostate cancer typically is diagnosed with a digital rectal exam (“DRE”) and/or prostate specific antigen (PSA) screening. An abnormal finding on DRE and/or an elevated serum PSA level (e.g., >4 ng/ml) can indicate the presence of prostate cancer. When a PSA or a DRE test is abnormal, a transrectal ultrasound may be used to map the prostate and show any suspicious areas. Biopsies of various sectors of the prostate are used to determine if prostate cancer is present.

The incidence increased with age and the routine availability of serum PSA testing has dramatically increased the number of aging men having the diagnosis. In most men the disease is slowly progressive but a significant number progress to metastatic disease which in time becomes androgen independent. Prognosis is good if the diagnosis is made when the cancer is still localized to the prostate; but nearly one-third of prostate cancers are diagnosed after the tumor has spread locally, and in 1 of 10 cases, the disease has distant metastases at diagnosis. The 5-year survival rate for men with advanced prostate cancer is only 33.6%. The choice of appropriate treatment is usually dependant on the age of the patient and the stage of the prostate cancer. This decision is complicated by the lack of available accurate methods to pre-surgically determine the clinical stage and the biologic potential of a given patient.

An important clinical question is how aggressively to treat such patients with localized prostate cancer. Usual treatment options depend on the stage of the prostate cancer. Men with a 10-year life expectancy or less who have a low Gleason number and whose tumor has not spread beyond the prostate often are not treated. Treatment options for more aggressive cancers include radical prostatectomy and/or radiation therapy. Androgen-depletion therapy (such as, gonadotropin-releasing hormone agonists (e.g., leuprolide, goserelin, etc.) and/or bilateral orchiectomy) is also used, alone or in conjunction with surgery or radiation. However, these prognostic indicators do not accurately predict clinical outcome for individual patients. Hence, critical understanding of the molecular abnormalities that define those tumors at high risk for relapse is needed to help identify more precise molecular markers.

Unlike many tumor types, specific patterns of oncogene expression have not been consistently identified in prostate cancer progression, although a number of candidate genes and pathways likely to be important in individual cases have been identified (Tomlins et al., Annu. Rev. Pathol. 1:243-71, 2006). Several groups have attempted to examine prostate cancer progression by comparing gene expression of primary carcinomas to normal prostate. Because of differences in technique as well as the true biologic heterogeneity seen in prostate cancer these studies have reported thousands of candidate genes but shared only moderate consensus. Nevertheless a few genes have emerged including hepsin (HPN) (Rhodes et al., Cancer Res. 62:4427-33, 2002), alpha-methylacyl-CoA racemase (AMACR) (Rubin et al., JAMA 287:1662-70, 2002), and enhancer of Zeste homolog 2 (EZH2) (Varambally et al., Nature 419:624-9, 2002), which have been shown experimentally to have probable roles on prostate carcinogenesis. Most recently, bioinformatics approaches and gene expression methods were used to identify fusion of the androgen-regulated transmembrane protease, serine 2 (TMPRSS2) with members of the erythroblast transformation specific (ETS) DNA transcription factors family (Tomlins et al., Science 310:644-8, 2005). This fusion appears commonly in prostate cancer and has been shown to be prevalent in more aggressive tumors (Attard et al., Oncogene 27:253-63, 2008; Demichelis et al., Oncogene 26:4596-9, 2007; Nam et al., Br. J. Cancer 97:1690-5, 2007). A number of studies have shown distinct classes of tumors separable by their gene expression (Rhodes et al., Cancer Res. 62:4427-33, 2002; Glinsky et al., J. Clin. Invest. 113:913-23, 2004; Lapointe et al., Proc. Natl. Acad. Sci. USA 101:811-6, 2004; Singh et al., Cancer Cell 1:203-9, 2002; Yu et al., J. Clin. Oncol. 22:2790-9, 2004), which may relate to the known clinical heterogeneity. A number of gene expression studies have been performed looking for gene dysregulation in metastatic versus primary prostate cancer (Varambally et al., Nature 419:624-9, 2002; Lapointe et al., Proc. Natl. Acad. Sci. USA 101:811-6, 2004; LaTulippe et al., Cancer Res. 62:4499-506, 2002).

Another factor impacting clinical utility of the various proposed panels is the fact that most samples availability for validation exist only as formalin fixed paraffin embedded (FFPE) tissues. In contrast, many of the cDNA microarray studies conducted to date typically use snap frozen tissues (Bibikova et al., Genomics 89:666-72, 2007; van't Veer et al., Nature 415:530-6, 2002). The ability to perform and analyze gene expression in FFPE tissues will greatly accelerate research by correlating already available clinical information such as histological grade and clinical stage with gene specific signatures.

Given that some prostate cancers need not be treated while others almost always are fatal and further given that the disease treatment can be unpleasant at best, there is a strong need for methods that allow care givers to predict the expected course of disease, including the likelihood of cancer recurrence, long-term survival of the patient, and the like, and to select the most appropriate treatment option accordingly.

### SUMMARY OF THE DISCLOSURE

Disclosed herein are gene signatures of prostate cancer recurrence, characterized at least in part by altered (e.g., increased or decreased) expression of one or more genes listed in Table 8, which characterizes prostate cancer in subjects afflicted with the disease. For example, gene expression of wingless-type MMTV integration site family member 5 (WNT5A), thymidine kinase 1 (TK1), and growth-arrest specific gene 1 (GAS1) and/or any other gene listed in Table 8 can be used to forecast prostate cancer outcome, e.g., disease recurrence or non-recurrence in patients who have (or are candidates for) prostatectomy. In particular examples, overexpression of WNT5A and TK1 and down-regulation of GAS1 indicates an increased likelihood that the prostate cancer will recur, and thus a poor prognosis. The disclosed gene signatures may be useful, for example, to screen prostate cancer patients for cancer recurrence, which can aid prognosis and the making of therapeutic decisions in prostate cancer. Methods and compositions (including kits) that embody this discovery are described.

### BRIEF DESCRIPTION OF THE FIGURES

FIGS. 1A-1D includes several panels relating to RNA recovery from formalin-fixed, paraffin-embedded (“FFPE”) tissue samples from patients with recurring or non-recurring prostate cancer. (A) shows a flow diagram generally outlining exemplary method steps from tissue recovery to RNA quantification. (B) shows a schematic for identifying and manually retrieving (using a Beecher punch) tissue cores (1.0 mm diameter, 2-5 mm length) from FFPE blocks for RNA isolation. (C) is a representative tissue slice stained with hematoxylin and eosin (“H&E”), which shows schematically where cancerous cells were identified by a pathologist and the tissue core isolated. (D) shows methods of RNA quality assessment used for the expression analysis described in Example 1. The Agilent BIOANALYZER™ electrophoresis RNA assay was conducted for all samples and traces were determined to be acceptable as a surrogate for RNA integrity. Real time PCR was conducted for the RPL13a housekeeping gene in all samples and dissociation curves indicated the presence of only one RNA species, which also was indicative of RNA quality suitable for further analysis. As a system control, the DASL™ assay was run for the Cancer DAP Analyses on freshly isolated RNA samples.

FIGS. 2A-2C includes several panels relating to DASL™ gene expression analyses of RNA isolated from FFPE tissue samples from patients with recurring or non-recurring prostate cancer. (A) Cluster analysis using rank invariant normalization for all evaluable genes (367) and all samples (24 prostate tests and 4 control breast specimens namely CTRL1-MCF7, CTRL2-Breast/MCF7, CTRL3-Breast 1 and CTRL4-Breast 2). The control breast cancer samples (freshly isolated RNA) clustered separately from the prostate cancer samples. Correlation (1-r) values are displayed on the axis. (B) Negative control sample plots show a significant number of RNA samples with signal >300, indicative of high test sample binding to irrelevant probe. (C) Cluster analysis only for samples with low background binding (p value for detection <0.05).

FIGS. 3A-C are a series of bar graphs showing differential expression of (A) WNT5A, (B) TK1 and (C) GAS1 between recurrent (n=4) and non-recurrent (n=5) groups for 9 samples. The average signal intensity between recurrent and non-recurrent groups for WNT5A: 2861.29 and 338.35; for TK1: 2156.17 and 752.25; and for GAS1 130.52 and 2387.13.

FIG. 4 is a ROC curve showing the performance of a logistic regression model that includes WNT5A, GAS1, and TK1 and was fit to the entire set of 27 samples. The area under the curve is 0.846, which indicates the model fits the data very well. Bootstrap re-sampling was used to improve the AUC estimates, using 100 randomly selected test cases. Vertical axis (Y-axis) indicates true positive rate (sensitivity) i.e., scoring of recurrent samples as recurrent; horizontal axis (X-axis) indicates false positive rate (1-specificity) i.e., scoring of non-recurrent samples as recurrent.

### Sequence Listing

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. All sequence database accession numbers referenced herein are understood to refer to the version of the sequence identified by that accession number as it was available on the filing date of this application. In the accompanying sequence listing:

SEQ ID NO: 1 is a human GAS1 nucleic acid (cDNA) sequence (CDS=residues 411-1448) (see, e.g., GENBANK™ Accession No. NM_{—}002048.1 (GI:4503918)).

SEQ ID NO: 2 is a human GAS1 amino acid sequence (see, e.g., GENBANK™ Accession No. NP_{—}002039.1 (GI:4503919))

SEQ ID NO: 3 is a nucleic acid sequence encoding human WNT5A (CDS=residues 319-1461) (see, e.g., GENBANK™ Accession No. NM_{—}003392.3 (GI:40806204)).

SEQ ID NO: 4 is a human WNT5A amino acid sequence (see, e.g., GENBANK™ Accession No. NP_{—}003383.2 (GI:40806205)).

SEQ ID NO: 5 is a human TK1 nucleic acid (cDNA) sequence (CDS=residues 85-915) (see, e.g., GENBANK™ Accession No. NM_{—}003258.3 (GI:155969679)).

SEQ ID NO: 6 is a human TK1 amino acid sequence (see, e.g., GENBANK™ Accession No. NP_{—}003249.2 (GI:155969680)).

SEQ ID NOs: 7 and 8 are forward and reverse primers, respectively, useful at least for qRT-PCR assays of RPL13a (OMIM Accession No. 113703; GENBANK™ Accession Nos. NM_{—}000977 (GI:15431296) (mRNA variant 1) and NM_{—}033251 (GI:15431294) (mRNA variant 2)).

SEQ ID NOs: 9-17 are exemplary Illumina probe sequences.

SEQ ID NOs: 18-21 are exemplary WNT5A primer sequences.

SEQ ID NOs: 22-23 are exemplary TK1 primer sequences.

SEQ ID NOs: 24-25 are exemplary GAS1 primer sequences.

### DETAILED DESCRIPTION

### I. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various disclosed embodiments, the following explanations of specific terms are provided:

Amplification of a nucleic acid molecule: Refers to methods used to increase the number of copies of a nucleic acid molecule, such as a WNT5A, TK1 or GAS1 nucleic acid molecule. The resulting products can be referred to as amplicons or amplification products. Methods of amplifying nucleic acid molecules are known in the art, and include MDA, PCR (such as RT-PCR and qRT-PCR), DOP-PCR, RCA, T7/Primase-dependent amplification, SDA, 3SR, NASBA, and LAMP, among others.

Cancer: Malignant neoplasm, for example one that has undergone characteristic anaplasia with loss of differentiation, increased rate of growth, invasion of surrounding tissue, and is capable of metastasis.

Complementary: A nucleic acid molecule is said to be “complementary” with another nucleic acid molecule if the two molecules share a sufficient number of complementary nucleotides to form a stable duplex or triplex when the strands bind (hybridize) to each other, for example by forming Watson-Crick, Hoogsteen or reverse Hoogsteen base pairs. Stable binding occurs when a nucleic acid molecule (e.g., nucleic acid probe or primer) remains detectably bound to a target nucleic acid sequence (e.g., WNT5A, TK1 or GAS1 target nucleic acid sequence) under the required conditions.

Complementarity is the degree to which bases in one nucleic acid molecule (e.g., nucleic acid probe or primer) base pair with the bases in a second nucleic acid molecule (e.g., target nucleic acid sequence). Complementarity is conveniently described by percentage, that is, the proportion of nucleotides that form base pairs between two molecules or within a specific region or domain of two molecules. For example, if 10 nucleotides of a 15 contiguous nucleotide region of a nucleic acid probe or primer form base pairs with a target nucleic acid molecule, that region of the probe or primer is said to have 66.67% complementarity to the target nucleic acid molecule.

In the present disclosure, “sufficient complementarity” means that a sufficient number of base pairs exist between one nucleic acid molecule or region thereof (such as a region of a probe or primer) and a target nucleic acid sequence (e.g., a WNT5a, TK1, or GAS1 nucleic acid sequence) to achieve detectable binding. A thorough treatment of the qualitative and quantitative considerations involved in establishing binding conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Contact: To bring one agent into close proximity to another agent, thereby permitting the agents to interact. For example, an antibody (or other specific binding agent) can be applied to a microscope slide or other surface containing a biological sample, thereby permitting detection of proteins (or protein-protein interactions or protein-nucleic acid interactions) in the sample that are specific for the antibody. In another example, a oligonucleotide probe or primer (or other nucleic acid binding agent) can be incubated with nucleic acid molecules obtained from a biological sample (and in some examples under conditions that permit amplification of the nucleic acid molecule), thereby permitting detection of nucleic acid molecules (or nucleic acid-nucleic acid interactions) in the sample that have sufficient complementarity to the probe or primer.

Detect: To determine if an agent (e.g., a nucleic acid molecule or protein) or interaction (e.g., binding between two proteins, between a protein and a nucleic acid, or between two nucleic acid molecules) is present or absent. In some examples this can further include quantification. In particular examples, an emission signal from a label is detected. Detection can be in bulk, so that a macroscopic number of molecules can be observed simultaneously. Detection can also include identification of signals from single molecules using microscopy and such techniques as total internal reflection to reduce background noise.

For example, use of an antibody specific for a particular protein (e.g., WNT5A, TK1 or GAS1) permits detection of the of the protein or protein-protein interaction in a sample, such as a sample containing prostate cancer tissue. In another example, use of a probe or primer specific for a particular gene (e.g., WNT5A, TK1 or GAS1) permits detection of the of the desired nucleic acid molecule in a sample, such as a sample containing prostate cancer tissue.

Diagnose: The process of identifying a medical condition or disease, for example from the results of one or more diagnostic procedures. In particular examples, diagnosis includes determining the prognosis of a subject, such as determining the likely outcome of a subject having a disease (e.g., prostate cancer) in the absence of additional therapy (e.g., life expectancy), for example predicting the likely recurrence of prostate cancer in a human subject after prostatectomy.

Differential Expression [of a nucleic acid sequence]: A nucleic acid sequence is differentially expressed when the amount of one or more of its expression products (e.g., transcript (e.g., mRNA) and/or protein) is higher or lower in one tissue (or cell) type as compared to another tissue (or cell) type. For example, a gene, e.g., WNT5A and/or TK1, the transcript or protein of which is more highly expressed in recurrent prostate cancer tissue (or cells) and less expressed in non-recurrent prostate cancer tissue (or cells) is differentially expressed. In another example, a gene, e.g., GAS1, the transcript or protein of which is more highly expressed in non-recurrent prostate cancer tissue (or cells) and less expressed in recurrent prostate cancer tissue (or cells) is differentially expressed.

Gene: A nucleic acid (e.g., genomic DNA, cDNA, or RNA) sequence that comprises coding sequences necessary for the production of a polypeptide, precursor, or RNA (e.g., mRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment is/are retained. The term also encompasses the coding region of a structural gene and the sequences located adjacent to the coding region on both the 5′ and 3′ ends for a distance of about 1 kb or more on either end such that the gene corresponds to the full-length mRNA. Sequences located 5′ of the coding region and present on the mRNA are referred to as 5′ untranslated sequences. Sequences located 3′ or downstream of the coding region and present on the mRNA are referred to as 3′ untranslated sequences. The gene as present in (or isolated from) a genome contains the coding regions (“exons”) interrupted with non-coding sequences termed “introns.” Introns are absent in the processed RNA (e.g., mRNA) transcript.

Gene expression: A multi-step process involving converting genetic information encoded in a genome and intervening nucleic acid sequences (e.g., mRNA) into a polypeptide. The genomic sequence of a gene is “transcribed” to produce RNA (e.g., mRNA, also referred to as a transcript). mRNA is “translated” to produce a corresponding protein. Gene expression can be regulated at many stages in the process. Increased or decreased gene expression can be detected by an increase or decrease, respectively, in any gene expression product (i.e., mRNA and/or protein). Increased or decreased gene expression can also be a result of genomic alterations, such as an amplification or deletion, respectively, of the region of the genome including the subject gene sequence.

Label: An agent capable of detection, for example by spectrophotometry, flow cytometry, or microscopy. For example, one or more labels can be attached to an antibody, thereby permitting detection of a target protein (such as WNT5A, TK1, or GAS1). Furthermore, one or more labels can be attached to a nucleic acid molecule, thereby permitting detection of a target nucleic acid molecule (such as WNT5A, TK1, or GAS1 DNA or RNA). Exemplary labels include radioactive isotopes, fluorophores, chromophores, ligands, chemiluminescent agents, enzymes, and combinations thereof.

Normal cells or tissue: Non-tumor, non-malignant cells and tissue.

Specific binding (or obvious derivations of such phrase, such as specifically binds, specific for, etc.): The particular interaction between one binding partner (such as a gene-specific probe or protein-specific antibody) and another binding partner (such as a target of a gene-specific probe or protein-specific antibody). Such interaction is mediated by one or, typically, more non-covalent bonds between the binding partners (or, often, between a specific region or portion of each binding partner). In contrast to non-specific binding sites, specific binding sites are saturable. Accordingly, one exemplary way to characterize specific binding is by a specific binding curve. A specific binding curve shows, for example, the amount of one binding partner (the first binding partner) bound to a fixed amount of the other binding partner as a function of the first binding partner concentration. As the first binding partner concentration increases under these conditions, the amount of the first binding partner bound will saturate. In another contrast to non-specific binding sites, specific binding partners involved in a direct association with each other (e.g., a probe-mRNA or antibody-protein interaction) can be competitively removed (or displaced) from such association by excess amounts of either specific binding partner. Such competition assays (or displacement assays) are very well known in the art.

Subject: Includes any multi-cellular vertebrate organism, such as human and non-human mammals (e.g., veterinary subjects). In some examples, a subject is one who has cancer, or is suspected of having cancer, such as prostate cancer.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including”; hence, “comprising A or B” means “including A” or “including B” or “including A and B.”

Suitable methods and materials for the practice and/or testing of embodiments of a disclosed invention are described below. Such methods and materials are illustrative only and are not intended to be limiting. Other methods and materials similar or equivalent to those described herein also can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

All sequences associated with the GenBank® accession numbers referenced herein are incorporated by reference (e.g., the sequence present on Sep. 15, 2008 is incorporated by reference).

### II. Prostate Cancer Biomarkers

Disclosed herein are genes (see, e.g., Table 8) the expression of which characterizes prostate cancer in subjects afflicted with the disease. Methods and compositions that embody this discovery are described.

### A. Methods of Use

This disclosure identifies a number of genes that are differentially expressed in recurrent versus non-recurrent prostate cancer. The recurrence of prostate cancer after treatment (e.g., prostatectomy) is indicative (at least) of a more-aggressive cancer, a worse prognosis for the patient, an increased likelihood of disease progression, failure (or inadequacy) of treatment, and/or a need for alternative (or additional) treatments. Accordingly, the present discoveries have enabled, among other things, a variety of methods for characterizing prostate cancer tissues, diagnosis or prognosis of prostate cancer patients, predicting treatment outcome in prostate cancer patients, and directing (e.g., selecting useful) treatment modalities for prostate cancer patients.

Disclosed methods can be performed using biological samples obtained from any subject having prostate cancer. A typical subject is a human male; however, any mammal that has a prostate that may develop cancer can serve as a source of a biological sample useful in a disclosed method. Exemplary biological samples useful in a disclosed method include tissue samples (such as, prostate biopsies and/or prostatectomy tissues) or prostate cell samples (such as can be collected by prostate massage, in the urine, or in fine needle aspirates). Samples may be fresh or processed post-collection (e.g., for archiving purposes). In some examples, processed samples may be fixed (e.g., formalin-fixed) and/or wax- (e.g., paraffin-) embedded. Fixatives for mounted cell and tissue preparations are well known in the art and include, without limitation, 95% alcoholic Bouin's fixative; 95% alcohol fixative; B5 fixative, Bouin's fixative, formalin fixative, Karnovsky's fixative (glutaraldehyde), Hartman's fixative, Hollande's fixative, Orth's solution (dichromate fixative), and Zenker's fixative (see, e.g., Carson, Histotechology: A Self-Instructional Text, Chicago: ASCP Press, 1997). Particular method embodiments involve FFPE prostate cancer tissue samples. In some examples, the sample (or a fraction thereof) is present on a solid support. Solid supports useful in a disclosed method need only bear the biological sample and, optionally, but advantageously, permit the convenient detection of components (e.g., proteins and/or nucleic acid sequences) in the sample. Exemplary supports include microscope slides (e.g., glass microscope slides or plastic microscope slides), coverslips (e.g., glass coverslips or plastic coverslips), tissue culture dishes, multi-well plates, membranes (e.g., nitrocellulose or polyvinylidene fluoride (PVDF)) or BIACORE™ chips.

Exemplary methods involve determining in a prostate tissue sample from a subject the expression level of one or more of the genes disclosed in Table 8. The gene(s) useful in a disclosed method include (or consist of) any individual gene in Table 8 (such as GAS1, WNT5A, or TK1), or any combination of two or more genes in Table 8 (e.g., any two, three, four, five, six, seven, eight, nine, 10, 12, 15, 20, 25, or all 33 of the genes in Table 8, or at least two, at least three, at least four, at least five, at least six, at least seven, at least eight, at least nine, at least 10, at least 12, at least 15, at least 20, or at least 25 of the genes in Table 8). In particular embodiments, a combination of genes selected from those in Table 8 includes GAS1, WNT5A, TK1, GAS1 and WNT5A, GAS1 and TK1, WNT5A and TK1, or GAS1, WNT5A and TK1. In more particular embodiments, genes useful in a disclosed method consist of two or more of GAS1, WNT5A, and TK1, in any combination (such as GAS1 and WNT5A, GAS1 and TK1, WNT5A and TK1, or GAS1, WNT5A and TK1). Genes of interest in other method embodiments include (or consist of) GAS1, WNT5A, TK1, E2F5, or MSH2, or any combination thereof.

In exemplary methods, expression of WNT5A and/or TK1 is increased and/or expression of GAS1 is decreased as compared to a standard value or a control sample. In other methods, the expression of another gene in Table 8 (i.e., a gene other than WNT5A, TK1 or GAS1, such as E2F5 and/or MSH2) is increased. In some such methods, the relative increased expression of WNT5A and/or TK1 (and/or another gene in Table 8, such as E2F5 and/or MSH2) and/or the relative decreased expression of GAS1 indicates, for example, a higher likelihood of prostate cancer progression in the subject, an increased likelihood that the prostate cancer will recur after surgery (e.g., prostatectomy), a poor prognosis for the patient from whom the sample is collected, and/or a higher likelihood that surgical treatment (e.g., prostatectomy) will fail, and an increased need for a non-surgical or alternate treatment for the prostate cancer.

In some methods, the expression of one or more genes of interest (e.g., WNT5A, TK1, and GAS1) is measured relative to a standard value or a control sample. A standard values can include, without limitation, the average expression of the one or more genes of interest in a normal prostate (e.g., calculated in an analogous manner to the expression value of the genes in the prostate cancer sample), the average expression of the one or more genes of interest in a prostate sample obtained from a patient or patient population in which it is known that prostate cancer did not recur post-surgery, or the average expression of the one or more genes of interest in a prostate sample obtained from a patient or patient population in which it is known that prostate cancer did recur post-surgery. A control sample can include, for example, normal prostate tissue or cells, prostate tissue or cells collected from a patient or patient population in which it is known that prostate cancer did not recur post-surgery, prostate tissue or cells collected from a patient or patient population in which it is known that prostate cancer did recur post-surgery, lymphocytes collected from the subject or prostate disease-free individuals, and/or cells collected by buccal swab of the subject or prostate disease-free individuals.

In other methods, expression of the gene(s) of interest is (are) measured in test (i.e., prostate cancer patient sample) and control samples relative to a value obtained for a housekeeping gene (e.g., one or more of GAPDH (glyceraldehyde 3-phosphate dehydrogenase), SDHA (succinate dehydrogenase), HPRT1 (hypoxanthine phosphoribosyl transferase 1), HBS1L (HBS1-like protein), β-actin, and AHSP (alpha haemoglobin stabilizing protein)) in each sample to produce normalized test and control values; then, the normalized value of the test sample is compared to the normalized value of the control sample to obtain the relative expression of the gene(s) of interest (e.g., increased or decreased expression).

An increase or decrease in gene expression may mean, for example, that the expression of a particular gene expression product (e.g., transcript (e.g., mRNA) or protein) in the test sample is at least about 1%, at least about 2%, at least about 5%, at least about 10%, at least about 15%, at least about 20%, at least about 25%, at least about 30%, at least about 50%, at least about 75%, at least about 100%, at least about 150%, or at least about 200% higher or lower, respectively, of the applicable control (e.g., standard value or control sample). Alternatively, relative expression (i.e., increase or decrease) may be in terms of fold difference; for example, the expression of a particular gene expression product (e.g., transcript (e.g., mRNA) or protein) in the test sample may be at least about 2 fold, at least about 3 fold, at least about 4 fold, at least about 5 fold, at least about 8 fold, at least about 10 fold, at least about 20 fold, at least about 50 fold, at least about 100 fold, or at least about 200 fold times higher or lower, respectively, of the applicable control (e.g., standard value or control sample).

In some method embodiments where protein expression as determined by immunohistochemistry is used as a measure of gene expression, scoring of protein expression may be semi-quantitative; for example, with protein expression levels recorded as 0, 1, 2, or 3 (including, in some instances plus (or minus) values at each level, e.g., 1+, 2+, 3+) with 0 being substantially no detectable protein expression and 3 (or 3+) being the highest detected protein expression. In such methods, an increase or decrease in the corresponding gene expression is measured as a difference in the score as compared the applicable control (e.g., standard value or control sample); that is, a score of 3+ in a test sample as compared to a score of 0 for the control represents increased gene expression in the test sample, and a score of 0 in a test sample as compared to a score of 3+ for the control represents decreased gene expression in the test sample.

Exemplary methods predict the likelihood of prostate cancer recurrence. Recurrence means the prostate cancer has returned after an initial (or subsequent) treatment(s). Representative initial treatments include radiation treatment, chemotherapy, anti-hormone treatment and/or surgery (e.g., prostatectomy). Typically after an initial prostate cancer treatment PSA levels in the blood decrease to a stable and low level and, in some instances, eventually become almost undetectable. In some examples, recurrence of the prostate cancer is marked by rising PSA levels (e.g., greater than 2.0-2.5 ng/mL) and/or by identification of prostate cancer cells in the blood, prostate biopsy or aspirate, in lymph nodes (e.g., in the pelvis or elsewhere) or at a metastatic site (e.g., muscles that help control urination, the rectum, the wall of the pelvis, in bones or other organs). Serum PSA levels may be characterized as follows (although some variation of the following ranges is common in the art):

| Normal Range | 0 to 2.5 | ng/mL |
|---|---|---|
| Slightly to Moderately | 2.6 to 10 | ng/mL |
| Elevated | | |
| Moderately Elevated | 10 to 19.9 | ng/mL |
| Significantly Elevated | 20 | ng/mL or more |

Other exemplary methods predict the likelihood of prostate progression. Prostate cancer progression means that one or more indices of prostate cancer (e.g., serum PSA levels) show that the disease is advancing independent of treatment. In some examples, prostate cancer progression is marked by rising PSA levels (e.g., greater than 2.0-2.5 ng/mL) and/or by identification of (or increasing numbers of) prostate cancer cells in the blood, prostate biopsy or aspirate, in lymph nodes (e.g., in the pelvis or elsewhere) or at a metastatic site (e.g., muscles that help control urination, the rectum, the wall of the pelvis, in bones or other organs).

An increased likelihood of prostate cancer progression or prostate cancer recurrence can be quantified by any known metric. For example, an increased likelihood means at least a 10% chance of occurring (such as at least a 25% chance, at least a 50% chance, at least a 60% chance, at least a 75% chance or even greater than an 80% chance of occurring).

Some method embodiments are useful for prostate cancer prognosis. Prognosis is the likely outcome of the disease (typically independent of treatment). The gene signature(s) disclosed herein predict prostate cancer recurrence in a sample collected well prior to such recurrence. Hence, such gene signature is a surrogate for the aggressiveness of the cancer with recurring cancers being more aggressive. A poor (or poorer) prognosis is likely for a subject with a more aggressive cancer. In some method embodiments, a poor prognosis is less than 5 year survival (such as less than 1 year survival or less than 2 year survival) of the patient after initial diagnosis of the neoplastic disease. In some method embodiments, a good prognosis is greater than 2-year survival (such as greater than 3-year survival, greater than 5-year survival, or greater than 7-year survival) of the patient after initial diagnosis of the neoplastic disease.

Still other method embodiments predict treatment outcome in prostate cancer patients, and are useful for directing (e.g., selecting useful) treatment modalities for prostate cancer patients. As discussed elsewhere in this specification, expression of the disclosed genes predicts that prostate cancer treatment (e.g., prostatectomy) is likely to fail (e.g., the disease will recur). Hence, the disclosed gene signature(s) can be used by caregivers to counsel prostate cancer patients as to the likely success of treatment (e.g., prostatectomy). Taken in the context of the particular subject's medical history, the patient and the caregiver can make better informed decisions of whether or not to treat (e.g., perform surgery, such as prostatectomy) and/or whether or not to provide alternate treatment (such as, external beam radiotherapy, brachytherapy, chemotherapy, or watchful waiting).

1. Determining Gene Expression Level (e.g., Gene Expression Profiling)

Gene expression levels may be determined in a disclosed method using any technique known in the art. Exemplary techniques include, for example, methods based on hybridization analysis of polynucleotides (e.g., genomic nucleic acid sequences and/or transcripts (e.g., mRNA)), methods based on sequencing of polynucleotides, methods based on detecting proteins (e.g., immunohistochemistry and proteomics-based methods).

As discussed previously, gene expression levels may be affected by alterations in the genome (e.g., gene amplification, gene deletion, or other chromosomal rearrangements or chromosome duplications (e.g., polysomy) or loss of one or more chromosomes). Accordingly, in some embodiments, gene expression levels may be inferred or determined by detecting such genomic alterations. Genomic sequences harboring genes of interest may be quantified, for example, by in situ hybridization of gene-specific genomic probes to chromosomes in a metaphase spread or as present in a cell nucleus. The making of gene-specific genomic probes is well known in the art (see, e.g., U.S. Pat. Nos. 5,447,841, 5,756,696, 6,872,817, 6,596,479, 6,500,612, 6,607,877, 6,344,315, 6,475,720, 6,132,961, 7,115,709, 6,280,929, 5,491,224, 5,663,319, 5,776,688, 5,663,319, 5,776,688, 6,277,569, 6,569,626, U.S. patent application Ser. No. 11/849,060, and PCT Appl. No. PCT/U.S.07/77444). In some exemplary methods, quantification of gene amplifications or deletions may be facilitated by comparing the number of binding sites for a gene-specific genomic probe to a control genomic probe (e.g., a genomic probe specific for the centromere of the chromosome upon which the gene of interest is located). In some examples, gene amplification or deletion may be determined by the ratio of the gene-specific genomic probe to a control (e.g., centromeric) probe. For example, a ratio greater than two (such as greater than three, greater than four, greater than five or ten or greater) indicates amplification of the gene (or the chromosomal region) to which the gene-specific probe binds. In another example, a ratio less than one indicates deletion of the gene (or the chromosomal region) to which the gene-specific probe binds. In particular method embodiments, it can be advantageous to also determine that gene amplification or deletion is accompanied by a corresponding increase or decrease, respectively, in the expression products of the gene (e.g., mRNA or protein); however, once a correlation is established, continued co-detection is not needed (and may consume unnecessary resources and time).

Gene expression levels also can be determined by quantification of gene transcript (e.g., mRNA). Commonly used methods known in the art for the quantification of mRNA expression in a sample include, without limitation, northern blotting and in situ hybridization (e.g., Parker and Barnes, Meth. Mol. Biol., 106:247-283, 1999)); RNAse protection assays (e.g., Hod, Biotechniques, 13:852-854, 1992); and PCR-based methods, such as reverse transcription polymerase chain reaction (RT-PCR) (Weis et al., Trends in Genetics, 8:263-264, 1992) and real time quantitative PCR, also referred to as qRT-PCR). Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE), and gene expression analysis by massively parallel signature sequencing (MPSS).

Some method embodiments involving the determination of mRNA levels utilize RNA (e.g., total RNA) isolated from a target sample, such a prostate cancer tissue sample. General methods for RNA (e.g., total RNA) isolation are well known in the art and are disclosed in standard textbooks of molecular biology, including Ausubel et al., Current Protocols of Molecular Biology, John Wiley and Sons (1997). Methods for RNA extraction from paraffin-embedded tissues are disclosed in Examples herein and, for example, by Rupp and Locker (Lab. Invest., 56:A67, 1987) and DeAndres et al. (BioTechniques, 18:42044, 1995). In particular examples, RNA isolation can be performed using a purification kit, buffer set and protease obtained from commercial manufacturers, such as Qiagen, according to the manufacturer's instructions. Other commercially available RNA isolation kits include MASTERPURE™ Complete DNA and RNA Purification Kit (EPICENTRE™ Biotechnologies) and Paraffin Block RNA Isolation Kit (Ambion, Inc.).

In the MassARRAY™ gene expression profiling method (Sequenom, Inc.), cDNA obtained from reverse transcription of total RNA is spiked with a synthetic DNA molecule (competitor), which matches the targeted cDNA region in all positions, except a single base, and serves as an internal standard. The cDNA/competitor mixture is amplified by standard PCR and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derived PCR products. After purification, these products are dispensed on a chip array, which is pre-loaded with components needed for analysis with matrix-assisted laser desorption ionization time-of-flight (MALDI-TOF) mass spectrometry analysis. The cDNA present in the reaction is then quantified by analyzing the ratios of the peak areas in the mass spectrum generated. For further details see, e.g., Ding and Cantor, Proc. Natl. Acad. Sci. USA, 100:3059-3064, 2003. Other methods for determining mRNA expression that involve PCR include, for example, differential display (Liang and Pardee, Science, 257:967-971, 1992)); amplified fragment length polymorphism (Kawamoto et al., Genome Res., 12:1305-1312, 1999); BEADARRAY™ technology (Illumina, San Diego, Calif., USA; Oliphant et al., Discovery of Markers for Disease (Supplement to Biotechniques), June 2002; Ferguson et al., Anal. Chem., 72:5618, 2000; and Examples herein); XMAP™ technology (Luminex Corp., Austin, Tex., USA); BADGE assay (Yang et al., Genome Res., 11:1888-1898, 2001)); and high-coverage expression profiling (HiCEP) analysis (Fukumura et al., Nucl. Acids. Res., 31(16):e94, 2003).

Differential gene expression also can be determined using microarray techniques. In these methods, specific binding partners, such as probes (including cDNAs or oligonucleotides) specific for RNAs of interest or antibodies specific for proteins of interest are plated, or arrayed, on a microchip substrate. The microarray is contacted with a sample containing one or more targets (e.g., mRNA or protein) for one or more of the specific binding partners on the microarray. The arrayed specific binding partners form specific detectable interactions (e.g., hybridized or specifically bind to) their cognate targets in the sample of interest.

Serial analysis of gene expression (SAGE) is a method that allows the simultaneous and quantitative analysis of a large number of gene transcripts, without the need of providing an individual hybridization probe for each transcript. In the SAGE method, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantified by determining the abundance of individual tags, and identifying the gene corresponding to each tag (see, e.g., Velculescu et al., Science, 270:484-487, 1995, and Velculescu et al., Cell, 88:243-51, 1997).

Gene expression analysis by massively parallel signature sequencing (MPSS) was first described by Brenner et al. (Nature Biotechnology, 18:630-634, 2000). It is a sequencing approach that combines non-gel-based signature sequencing with in vitro cloning of millions of templates on separate 5 μm diameter microbeads. A microbead library of DNA templates is constructed by in vitro cloning. This is followed by the assembly of a planar array of the template-containing microbeads in a flow cell at a high density. The free ends of the cloned templates on each microbead are analyzed simultaneously using a fluorescence-based signature sequencing method that does not require DNA fragment separation.

In some examples, differential gene expression is determined using in situ hybridization techniques, such as fluorescence in situ hybridization (FISH) or chromogen in situ hybridization (CISH). In these methods, specific binding partners, such as probes labeled with a flouorphore or chromogen specific for a target cDNA or mRNA (e.g., a GAS1, TK1, or WNT5A cDNA or mRNA molecule) is contacted with a sample, such as a prostate cancer sample mounted on a substrate (e.g., glass slide). The specific binding partners form specific detectable interactions (e.g., hybridized to) their cognate targets in the sample. For example, hybridization between the probes and the target nucleic acid can be detected, for example by detecting a label associated with the probe. In some examples, microscopy, such as fluorescence microscopy, is used.

Immunohistochemistry (IHC) is one exemplary technique useful for detecting protein expression products in the disclosed methods. Antibodies (e.g., monoclonal and/or polyclonal antibodies) specific for each protein expression marker are used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available.

Proteomic analysis is another exemplary technique useful for detecting protein expression products in the disclosed methods. The term “proteome” is defined as the totality of the proteins present in a sample (e.g., tissue, organism, or cell culture) at a certain point of time. Proteomics includes, among other things, study of the global changes of protein expression in a sample (also referred to as “expression proteomics”). An exemplary proteomics assay involves (i) separation of individual proteins in a sample, e.g., by 2-D gel electrophoresis; (ii) identification of the individual proteins recovered from the gel, e.g., by mass spectrometry or N-terminal sequencing, and (iii) analysis of the data.

### B. Exemplary Prostate Cancer Biomarkers

1. Growth Arrest-Specific 1 (GAS1)

The human Growth Arrest-Specific 1 (GAS1) gene is located on chromosome 9 at gene map locus 9q21.3-q22.1 and encodes a 45 kDa glycophosphatydlinositol (GPI)-linked protein. Exemplary GAS1 sequences are publically available, for example from GenBank® (e.g., accession numbers NP_{—}002039.2 and AAH55747.1 (proteins) and BC132682.1 and NM_{—}008086.1 (cDNAs)). GAS1 protein (see, e.g., SEQ ID NO: 2) is a putative tumor suppressor. It plays a role in growth suppression (Del Sal et al., Cell, 70:595-607, 1992). In particular, GAS1 blocks entry to S phase and prevents cycling of normal and transformed cells. GAS1 is related to the GDNFα receptors and regulates Ret signaling (Cabrera et al., J. Biol. Chem., 281(20):14330-9, 2006).

Del Sal et al. (Proc. Nat. Acad. Sci. USA, 91:1848-1852, 1994) cloned human GAS1 cDNA (see, e.g., SEQ ID NO: 1). The derived 345-amino acid protein contained 2 putative transmembrane domains, an RGD consensus recognition sequence, and 1 potential N-glycosylation site. Stebel et al. (FEBS Lett., 481:152-8, 2000) demonstrated that the GAS1 protein undergoes co-translational modifications, including signal peptide cleavage, N-linked glycosylation, and glycosylphosphatidylinositol anchor addition.

Del Sal et al. (Proc. Nat. Acad. Sci. USA, 91:1848-1852, 1994) demonstrated that overexpression of the human GAS1 gene blocks cell proliferation in lung and bladder carcinoma cell lines, but not in an osteosarcoma cell line or in an adenovirus-type-5 transformed cell line. Del Sal et al. (Cell, 70:595-607, 1992) had previously shown that SV40-transformed NIH 3T3 cells also are refractory to murine GAS1 overexpression, suggesting that the retinoblastoma and/or p53 gene products have an active role in mediating the growth-suppressing effect of GAS1. Martinelli and Fan (Genes Dev., 21:1231-1243, 2007) found that GAS1 positively regulated hedgehog signaling in developing mouse and chicken, an effect particularly noticeable at regions where hedgehog acted at low concentrations.

Seppala et al. (J. Clin. Invest., 117:1575-1584, 2007) generated GAS1 −/− mice and observed microform holoprosencephaly, including midfacial hypoplasia, premaxillary incisor fusion, and cleft palate, in addition to severe ear defects; however, the forebrain remained grossly intact. These defects were associated with a loss of Shh signaling in cells at a distance from the source of transcription.

2. Wingless-Type MMMTV Integration Site Family, Member 5A (WNT5A)

The human WNT5A gene is located on chromosome 3 at gene map locus 3p21-p14. The Wnt genes belong to a family of protooncogenes with at least 13 known members that are expressed in species ranging from Drosophila to man. The Wnts are lipid-modified secreted glycoproteins that regulate diverse biologic functions including roles in developmental patterning, cell proliferation, differentiation, cell polarity, and morphogenetic movement (Logan and Nusse, Annu. Rev. Cell. Dev. Biol. 20:781-810, 2004). Transcription of Wnt family genes appears to be developmentally regulated in a precise temporal and spatial manner.

Gavin et al. (Genes Dev., 4:2319-2332, 1990) identified 6 new members of the Wnt gene family, including WNT5A, in the mouse. The Wnt genes encode 38- to 43-kD Cys-rich putative glycoproteins, which have features typical of secreted growth factors (e.g., a hydrophobic signal sequence and 21 conserved cysteine residues whose relative spacing is maintained) (see, e.g., SEQ ID NO: 4).

Clark et al. (Genomics, 18:249-260, 1993) cloned the human Wnt5A cDNA (see, e.g., SEQ ID NO: 3). Other exemplary WNT5A sequences are publically available, for example from GenBank® (e.g., accession numbers AAH74783.2 and AAV69750.1 (proteins) and NM_{—}003392.3 and NM_{—}009524.2 (cDNAs)). He et al. (Science, 275:1652-1654, 1997) showed that human frizzled-5 is the receptor for WNT5A. The Wnt ligands utilize receptors of the Frizzle family and signaling is usually divided into two pathways: the ‘canonical pathway’ which acts through beta-catenin, and the ‘non-canonical pathway’ acting through the Ca²⁺ and planar polarity pathways (Veeman et al., Dev. Cell 5:367-77, 2003). WNT5A protein has been shown to influence transcription by effecting histone methylation, increase cell migration, influence cell polarity, induce endothelial proliferation, and increase expression of certain metalloproteinases.

3. Soluble Thymidine Kinase (TK1)

The human TK1 gene is located on chromosome 17 at gene map locus 17q25.2-q25.3. For exemplary cDNA and protein sequences see SEQ ID NOs: 5 and 6, respectively. Other exemplary TK1 sequences are publically available, for example from GenBank® (e.g., accession numbers NP_{—}003249.3 and NP_{—}033413.1 (proteins) and AB451268.1 and NM_{—}052800.1 (cDNAs)).

Thymidine kinase (EC 2.7.1.21) catalyzes the phosphorylation of thymidine to deoxythymidine monophosphate. Lin et al. (Proc. Nat. Acad. Sci. USA, 80:6528-6532, 1983) cloned the TK1 gene and estimated its maximal size to be 14 kb and its minimal size between 4 and 5 kb. The gene contains many noncoding inserts and numerous Alu sequences. Sherley and Kelly (J. Biol. Chem., 263:375-382, 1988) purified and characterized the enzyme from HeLa cells. In the 5′ flanking region of the TK gene, Sauve et al. (DNA Sequence, 1:13-23, 1990) located the position of nucleotide sequences that can act as binding sites for trans-acting factors as well as potential cis-acting sequences. The latter were compared with those of the promoter of the human proliferating cell nuclear antigen (PCNA) gene. Both TK and PCNA are maximally expressed at the G1/S boundary of the cell cycle.

4. Variant Sequences

In addition to the specific sequences provided herein, and the sequences which are currently publically available, one skilled in the art will appreciate that variants of such sequences may be present in a particular subject. For example, polymorphisms for a particular gene or protein may be present. In addition, a sequence may vary between different organisms. In particular examples, a variant sequence retains the biological activity of its corresponding native sequence. For example, a sequence present in a particular subject (e.g., a WNT5A, TK1, or GAS1 sequence or any other gene/protein listed in Table 8) may can have conservative amino acid changes (such as, very highly conserved substitutions, highly conserved substitutions or conserved substitutions), such as 1 to 5 or 1 to 10 conservative amino acid substitutions. Exemplary conservative amino acid substitutions are shown in Table 1.

TABLE 1
Exemplary conservative amino acid
substitutions.

| | Very | | |
|---|---|---|---|
| | Highly- | Highly Conserved | Conserved |
| | Conserved | Substitutions | Substitutions |
| Original | Substi- | (from the | (from the |
| Residue | tutions | Blosum90 Matrix) | Blosum65 Matrix) |
| Ala | Ser | Gly, Ser, Thr | Cys, Gly, Ser, |
| | | | Thr, Val |
| Arg | Lys | Gln, His, Lys | Asn, Gln, Glu, |
| | | | His, Lys |
| Asn | Gln; His | Asp, Gln, His, | Arg, Asp, Gln, |
| | | Lys, Ser, Thr | Glu, His, Lys, |
| | | | Ser, Thr |
| Asp | Glu | Asn, Glu | Asn, Gln, Glu, |
| | | | Ser |
| Cys | Ser | None | Ala |
| Gln | Asn | Arg, Asn, Glu, | Arg, Asn, Asp, |
| | | His, Lys, Met | Glu, His, Lys, |
| | | | Met, Ser |
| Glu | Asp | Asp, Gln, Lys | Arg, Asn, Asp, |
| | | | Gln, His, Lys, |
| | | | Ser |
| Gly | Pro | Ala | Ala, Ser |
| His | Asn; Gln | Arg, Asn, Gln, | Arg, Asn, Gln, |
| | | Tyr | Glu, Tyr |
| Ile | Leu; Val | Leu, Met, Val | Leu, Met, Phe, |
| | | | Val |
| Leu | Ile; Val | Ile, Met, Phe, | Ile, Met, Phe, |
| | | Val | Val |
| Lys | Arg; Gln; | Arg, Asn, Gln, | Arg, Asn, Gln, |
| | Glu | Glu | Glu, Ser, |
| Met | Leu; Ile | Gln, Ile, Leu, | Gln, Ile, Leu, |
| | | Val | Phe, Val |
| Phe | Met; Leu; | Leu, Trp, Tyr | Ile, Leu, Met, |
| | Tyr | | Trp, Tyr |
| Ser | Thr | Ala, Asn, Thr | Ala, Asn, Asp, |
| | | | Gln, Glu, Gly, |
| | | | Lys, Thr |
| Thr | Ser | Ala, Asn, Ser | Ala, Asn, Ser, |
| | | | Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, |
| | | | Met, Thr |

In some embodiments, a WNT5A, TK1, or GAS1 sequence is a sequence variant of a native WNT5A, TK1, or GAS1 sequence, respectively, such as a nucleic acid or protein sequence that has at least 99%, at least 98%, at least 95%, at least 92%, at least 90%, at least 85%, at least 80%, at least 75%, at least 70%, at least 65%, or at least 60% sequence identity to the sequences set forth in SEQ ID NOS: 1-6 (or such amount of sequence identity to a GenBank® accession number referred to herein) wherein the resulting variant retains WNT5A, TK1, or GAS1 biological activity. “Sequence identity” is a phrase commonly used to describe the similarity between two amino acid sequences (or between two nucleic acid sequences). Sequence identity typically is expressed in terms of percentage identity; the higher the percentage, the more similar the two sequences.

In particular examples, a sequence variant of a gene or protein listed in Table 8 has one or more conservative amino acid substitutions as compared to a native sequence or has a particular percentage sequence identity (e.g., at least 99%, at least 98%, at least 95%, at least 92%, at least 90%, at least 85%, at least 80%, at least 75%, at least 70%, at least 65%, or at least 60% sequence identity) to a native sequence. In particular examples, such a variant retains a significant amount of the biological activity of the native protein or nucleic acid molecule.

Methods for aligning sequences for comparison and determining sequence identity are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. USA, 85:2444, 1988; Higgins and Sharp, Gene, 73:237-244, 1988; Higgins and Sharp, CABIOS, 5:151-153, 1989; Corpet et al., Nucleic Acids Research, 16:10881-10890, 1988; Huang, et al., Computer Applications in the Biosciences, 8:155-165, 1992; Pearson et al., Methods in Molecular Biology, 24:307-331, 1994; Tatiana et al., FEMS Microbiol. Lett., 174:247-250, 1999. Altschul et al. present a detailed consideration of sequence-alignment methods and homology calculations (J. Mol. Biol., 215:403-410, 1990).

The National Center for Biotechnology Information (NCBI) Basic Local Alignment Search Tool (BLAST™, Altschul et al., J. Mol. Biol., 215:403-410, 1990) is publicly available from several sources, including the National Center for Biotechnology Information (NCBI, Bethesda, Md.) and on the Internet, for use in connection with the sequence-analysis programs blastp, blastn, blastx, tblastn and tblastx. A description of how to determine sequence identity using this program is available on the internet under the help section for BLASTT™.

For comparisons of amino acid sequences of greater than about 15 amino acids, the “Blast 2 sequences” function of the BLAST™ (Blastp) program is employed using the default BLOSUM62 matrix set to default parameters (cost to open a gap [default=5]; cost to extend a gap [default=2]; penalty for a mismatch [default=3]; reward for a match [default=1]; expectation value (E) [default=10.0]; word size [default=3]; and number of one-line descriptions (V) [default=100]. When aligning short peptides (fewer than around 15 amino acids), the alignment should be performed using the Blast 2 sequences function “Search for short nearly exact matches” employing the PAM30 matrix set to default parameters (expect threshold=20000, word size=2, gap costs: existence=9 and extension=1) using composition-based statistics.

### C. Compositions

Disclosed herein are genes (see, e.g., Table 8) the expression of which characterizes prostate cancer in subjects afflicted with the disease. Accordingly, compositions that facilitate the detection of such genes in biological samples are now enabled.

1. Kits

Kits useful for facilitating the practice of a disclosed method are also contemplated. In one embodiment, a kit is provided for detecting one or more of the genes disclosed in Table 8 (such as, at least one, at least two, at least three, at least five, at least seven, or at least ten of the genes disclosed in Table 8). In a specific example, kits are provided for detecting at least WNT5A, TK1, and GAS1 nucleic acid or protein molecules, for example in combination with one to ten (e.g., 1, 2, 3, 4, or 5) housekeeping genes or proteins (e.g., β-actin, GAPDH, SDHA, HPRT1, HBS1L, and AHSP). In yet other specific examples, kits are provided for detecting only WNT5A, TK1, and GAS1 nucleic acid or protein molecules. The detection means can include means for detecting a genomic alteration involving the gene and/or a gene expression product, such as an mRNA or protein. In particular examples, means for detecting one or more of the genes or proteins listed in Table 8 (such as means for detecting at least WNT5A, TK1, and GAS1) are packaged in separate containers or vials. In some examples, means for detecting one or more of the genes or proteins listed in Table 8 (such as means for detecting at least WNT5A, TK1, and GAS1) are present on an array (discussed below).

Exemplary kits can include at least one means for detection of one or more of the disclosed genes or gene products (such as, at least two, at least three, at least four, or at least five detection means), such as means that permit detection of at least WNT5A, TK1, and GAS1. In some examples, such kits can further include at least one means for detection of one or more (e.g., one to three) housekeeping genes or proteins. Detection means can include, without limitation, a nucleic acid probe specific for a genomic sequence including a disclosed gene, a nucleic acid probe specific for a transcript (e.g., mRNA) encoded by a disclosed gene, a pair of primers for specific amplification of a disclose gene (e.g., genomic sequence or cDNA sequence of such gene), an antibody or antibody fragment specific for a protein encoded by a disclosed gene. Particular kit embodiments can include, for instance, one or more (such as two, three, or four) detection means selected from a nucleic acid probe specific for WNT5A transcript, a nucleic acid probe specific for TK1 transcript, a nucleic acid probe specific for GAS1 transcript, a pair of primers for specific amplification of WNT5A transcript, a pair of primers for specific amplification of TK1 transcript, a pair of primers for specific amplification of GAS1 transcript, an antibody specific for WNT5A protein, an antibody specific for specific for TK1 protein, and an antibody specific for a GAS1 protein. Particular kit embodiments can further include, for instance, one or more (such as two or three) detection means selected from a nucleic acid probe specific for a housekeeping transcript, a pair of primers for specific amplification of housekeeping transcript, and an antibody specific for housekeeping protein. Exemplary housekeeping genes/proteins include GAPDH, SDHA, HPRT1, HBS1L, β-actin, and AHSP.

In some kit embodiments, the primary detection means (e.g., nucleic acid probe, nucleic acid primer, or antibody) can be directly labeled, e.g., with a fluorophore, chromophore, or enzyme capable of producing a detectable product (such as alkaline phosphates, horseradish peroxidase and others commonly know in the art). Other kit embodiments will include secondary detection means; such as secondary antibodies (e.g., goat anti-rabbit antibodies, rabbit anti-mouse antibodies, anti-hapten antibodies) or non-antibody hapten-binding molecules (e.g., avidin or streptavidin). In some such instances, the secondary detection means will be directly labeled with a detectable moiety. In other instances, the secondary (or higher order) antibody will be conjugated to a hapten (such as biotin, DNP, and/or FITC), which is detectable by a detectably labeled cognate hapten binding molecule (e.g., streptavidin (SA) horseradish peroxidase, SA alkaline phosphatase, and/or SA QDot™). Some kit embodiments may include colorimetric reagents (e.g., DAB, and/or AEC) in suitable containers to be used in concert with primary or secondary (or higher order) detection means (e.g., antibodies) that are labeled with enzymes for the development of such colorimetric reagents.

In some embodiments, a kit includes positive or negative control samples, such as a cell line or tissue known to express or not express a particular gene or gene product listed in Table 8. In particular examples, control samples are FFPE. Exemplary samples include but are not limited to normal (e.g., non cancerous) cells or tissues), breast cancer cell lines or tissues, prostate cancer samples from subject known not to have had prostate cancer recurrence following prostatectomy (e.g., at least 5 years or at least 10 years following prostatectomy), and prostate cancer samples from subject known to have had prostate cancer recurrence following prostatectomy.

In some embodiments, a kit includes instructional materials disclosing, for example, means of use of a probe or antibody that specifically binds a disclosed gene or its expression product (e.g., mRNA or protein), or means of use for a particular primer or probe. The instructional materials may be written, in an electronic form (e.g., computer diskette or compact disk) or may be visual (e.g., video files). The kits may also include additional components to facilitate the particular application for which the kit is designed. Thus, for example, the kit can include buffers and other reagents routinely used for the practice of a particular disclosed method. Such kits and appropriate contents are well known to those of skill in the art.

Certain kit embodiments can include a carrier means, such as a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, which packaging unit may have compartments into which one or more components of the kit can be placed. In other examples, a kit includes a one or more containers, for instance vials, tubes, and the like that can retain, for example, one or more biological samples to be tested.

Other kit embodiments include, for instance, syringes, cotton swabs, or latex gloves, which may be useful for handling, collecting and/or processing a biological sample. Kits may also optionally contain implements useful for moving a biological sample from one location to another, including, for example, droppers, syringes, and the like. Still other kit embodiments may include disposal means for discarding used or no longer needed items (such as subject samples, etc.). Such disposal means can include, without limitation, containers that are capable of containing leakage from discarded materials, such as plastic, metal or other impermeable bags, boxes or containers.

2. Arrays

Microarrays for the detection of genes (e.g., genomic sequence and corresponding transcripts) and proteins are well known in the art. Microarrays include a solid surface (e.g., glass slide) upon which many (e.g., hundreds or even thousands) of specific binding agents (e.g., cDNA probes, mRNA probes, or antibodies) are immobilized. The specific binding agents are distinctly located in an addressable (e.g., grid) format on the array. The number of addressable locations on the array can vary, for example from at least three, to at least 10, at least 20, at least 30, at least 33, at least 40, at least 50, at least 75, at least 100, at least 150, at least 200, at least 300, at least 500, least 550, at least 600, at least 800, at least 1000, at least 10,000, or more. The array is contacted with a biological sample believed to contain targets (e.g., mRNA, cDNA, or protein, as applicable) for the arrayed specific binding agents. The specific binding agents interact with their cognate targets present in the sample. The pattern of binding of targets among all immobilized agents provides a profile of gene expression. In particular embodiments, various scanners and software programs can be used to profile the patterns of genes that are “turned on” (e.g., bound to an immobilized specific binding agent). Representative microarrays are described, e.g., in U.S. Pat. Nos. 5,412,087, 5,445,934, 5,744,305, 6,897,073, 7,247,469, 7,166,431, 7,060,431, 7,033,754, 6,998,274, 6,942,968, 6,890,764, 6,858,394, 6,770,441, 6,620,584, 6,544,732, 6,429,027, 6,396,995, and 6,355,431.

Disclosed herein are arrays, whether protein or nucleic acid arrays, for the detection at least three of the genes (or gene-products) disclosed in Table 8. In particular embodiments, disclosed arrays consist of binding agents specific for at least four, at least five, at least 10, at least 15, at least 20, at least 25 or all 33 of the disclosed genes. Particular array embodiments consist of nucleic probes or antibodies specific for GAS1, WNT5A, TK1, E2F5, and MSH2 expression products (e.g., mRNA, cDNA or protein). More particular array embodiments consist of nucleic probes or antibodies specific for GAS1, WNT5A, and TK1 expression products (e.g., mRNA, cDNA or protein). Other array embodiments consist of nucleic probes or antibodies specific for expression products (e.g., mRNA, cDNA or protein) for each one of the 33 genes in Table 8; thus, an array consisting of nucleic probes or antibodies specific for mRNA, cDNA or protein, corresponding to all of the following genes: CDC25C, E2F5, MMP3, CYP1A1, FGF8, WNT5A, CHEK1, CSF2, CDC2, IL1A, ALK, MYBL2, MYCL1, MYCN, TERT, ALOX12, BRCA2, FANCA, GAS1, LMO1, PLG, TDGF1, TK1, BLM, MSH2, NAT2, DMBT1, FLT3, GFI1, MOS, TP73, HMMR, and INHA. In particular examples, the array further includes nucleic probes or antibodies specific for a housekeeping gene or gene product, such as mRNA, cDNA or protein,

a. Nucleic Acid Arrays

In one example, the array includes nucleic acid probes that can hybridize to at least three the genes listed in Table 8, such as at least four, at least five, at least 10, at least 15, at least 20, at least 25 or all 33 of the disclosed genes, for example includes nucleic acid probes that can hybridize to at least WNT5A, TK1, and GAS1 (e.g., includes probes that can hybridize to SEQ ID NO: 1, 3 or 5 or its complementary strand). In particular examples, an array includes probes that can recognize all 33 genes listed in Table 8. Certain of such arrays (as well as the methods described herein) can further include oligonucleotides specific for housekeeping genes (e.g., one or more of GAPDH (glyceraldehyde 3-phosphate dehydrogenase), SDHA (succinate dehydrogenase), HPRT1 (hypoxanthine phosphoribosyl transferase 1), HBS1L (HBS1-like protein), β-actin, and AHSP (alpha haemoglobin stabilizing protein)).

In one example, a set of oligonucleotide probes is attached to the surface of a solid support for use in detection of at least three of the genes listed in Table 8 (e.g., at least WNT5A, TK1, and GAS1), such as detection of nucleic acid sequences (such as cDNA or mRNA) obtained from the subject (e.g., from a prostate cancer sample). Additionally, if an internal control nucleic acid sequence is used (such as a nucleic acid sequence obtained from a subject who has not had a recurring prostate cancer or a housekeeping gene nucleic acid sequence) a nucleic acid probe can be included to detect the presence of this control nucleic acid molecule.

The oligonucleotide probes bound to the array can specifically bind sequences obtained from the subject, or amplified from the subject, such as under high stringency conditions. Agents of use with the method include oligonucleotide probes that recognize target gene sequences listed in Table 8. Such sequences can be determined by examining the known gene sequences, and choosing probe sequences that specifically hybridize to a particular gene listed in Table 8, but not other gene sequences.

The methods and apparatus in accordance with the present disclosure take advantage of the fact that under appropriate conditions oligonucleotide probes form base-paired duplexes with nucleic acid molecules that have a complementary base sequence. The stability of the duplex is dependent on a number of factors, including the length of the oligonucleotide probe, the base composition, and the composition of the solution in which hybridization is effected. The effects of base composition on duplex stability can be reduced by carrying out the hybridization in particular solutions, for example in the presence of high concentrations of tertiary or quaternary amines. The thermal stability of the duplex is also dependent on the degree of sequence similarity between the sequences. By carrying out the hybridization at temperatures close to the anticipated Tₘ's of the type of duplexes expected to be formed between the target sequences and the oligonucleotides bound to the array, the rate of formation of mis-matched duplexes may be substantially reduced.

The length of each oligonucleotide probe employed in the array can be selected to optimize binding of target sequences. An optimum length for use with a particular gene sequence under specific screening conditions can be determined empirically. Thus, the length for each individual element of the set of oligonucleotide sequences including in the array can be optimized for screening. In one example, oligonucleotide probes are at least 12 nucleotides in length, such as from about 20 to about 35 nucleotides in length or about 25 to about 40 nucleotides in length.

The oligonucleotide probe sequences forming the array can be directly linked to the support. Alternatively, the oligonucleotide probes can be attached to the support by oligonucleotides (that do not non-specifically hybridize to the target gene sequences) or other molecules that serve as spacers or linkers to the solid support.

b. Protein Arrays

In another example, an array includes protein sequences (or a fragment of such proteins, or antibodies specific to such proteins or protein fragments), which include at least three of the protein sequences listed in Table 3, such as at least four, at least five, at least 10, at least 15, at least 20, at least 25 or all 33 of the disclosed proteins, for example includes protein binding agents that can specifically bind to at least WNT5A, TK1, and GAS1 (e.g., can stably bind to SEQ ID NO: 2, 4 or 6, respectively). In particular examples, an array includes protein binding agents that can recognize all 33 proteins listed in Table 8. Certain of such arrays (as well as the methods described herein) can further include protein binding agents specific for housekeeping proteins (e.g., one or more of GAPDH, SDHA, HPRT1, HBS1L, β-actin, and AHSP).

The proteins or antibodies forming the array can be directly linked to the support. Alternatively, the proteins or antibodies can be attached to the support by spacers or linkers to the solid support. Changes in protein expression can be detected using, for instance, a protein-specific binding agent, which in some instances is labeled. In certain examples, detecting a change in protein expression includes contacting a protein sample obtained from a prostate cancer sample of a subject with a protein-specific binding agent (which can be for example present on an array); and detecting whether the binding agent is bound by the sample and thereby measuring the levels of the target protein present in the sample. A difference in the level of a target protein in the sample (e.g., WNT5A, TK1 and GAS1), relative to the level of the same target protein found an analogous sample from a subject who has not had a recurring prostate cancer, in particular examples indicates that the subject has a poor prognosis.

c. Array Substrate

The array solid support can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof (e.g., U.S. Pat. No. 5,985,567).

In general, suitable characteristics of the material that can be used to form the solid support surface include: being amenable to surface activation such that upon activation, the surface of the support is capable of covalently attaching a biomolecule such as an oligonucleotide or antibody thereto; amenability to “in situ” synthesis of biomolecules; being chemically inert such that at the areas on the support not occupied by the oligonucleotides or antibodies are not amenable to non-specific binding, or when non-specific binding occurs, such materials can be readily removed from the surface without removing the oligonucleotides or antibodies.

In one example, the solid support surface is polypropylene. Polypropylene is chemically inert and hydrophobic. Non-specific binding is generally avoidable, and detection sensitivity is improved. Polypropylene has good chemical resistance to a variety of organic acids (such as formic acid), organic agents (such as acetone or ethanol), bases (such as sodium hydroxide), salts (such as sodium chloride), oxidizing agents (such as peracetic acid), and mineral acids (such as hydrochloric acid). Polypropylene also provides a low fluorescence background, which minimizes background interference and increases the sensitivity of the signal of interest.

In another example, a surface activated organic polymer is used as the solid support surface. One example of a surface activated organic polymer is a polypropylene material aminated via radio frequency plasma discharge. Such materials are easily utilized for the attachment of nucleic acid molecules. The amine groups on the activated organic polymers are reactive with nucleotide molecules such that the nucleotide molecules can be bound to the polymers. Other reactive groups can also be used, such as carboxylated, hydroxylated, thiolated, or active ester groups.

d. Array Formats

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (e.g., U.S. Pat. No. 5,981,185). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. Particularly disclosed for preparation of arrays are biaxially oriented polypropylene (BOPP) films; in addition to their durability, BOPP films exhibit a low background fluorescence.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (e.g., see U.S. Pat. No. 6,013,789). In another example, sequences are synthesized directly onto the support to provide the desired array (e.g., see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (e.g., see PCT applications WO 85/01051 and WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second (2° set of rows that are now perpendicular to the first set. This process creates a multiple-channel array whose intersection generates a plurality of discrete cells.

Oligonucleotide probes can be bound to the support by either the 3′ end of the oligonucleotide or by the 5′ end of the oligonucleotide. In one example, the oligonucleotides are bound to the solid support by the 3′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support. In particular examples, the oligonucleotide probes on the array include one or more labels, that permit detection of oligonucleotide probe:target sequence hybridization complexes.

3. Protein Specific Binding Agents

In some examples, the means used to detect one or more (such as at least three) of the genes or gene products listed in Table 8 is a protein specific binding agent, such as an antibody or fragment thereof. For example, antibodies or aptamers specific for the proteins listed in Table 8, such as WNT5A, TK1, or GAS1 (e.g., SEQ ID NO: 2, 4, or 6, respectively), can be obtained from a commercially available source or prepared using techniques common in the art. Such specific binding agents can also be used in the prognostic methods provided herein.

Specific binding reagents include, for example, antibodies or functional fragments or recombinant derivatives thereof, aptamers, mirror-image aptamers, or engineered nonimmunoglobulin binding proteins based on any one or more of the following scaffolds: fibronectin (e.g., ADNECTINST™ or monobodies), CTLA-4 (e.g., EVIBODIES™), tendamistat (e.g., McConnell and Hoess, J. Mol. Biol., 250:460-470, 1995), neocarzinostatin (e.g., Heyd et al., Biochem., 42:5674-83, 2003), CBM4-2 (e.g., Cicortas-Gunnarsson et al., Protein Eng. Des. Sel., 17:213-21, 2004), lipocalins (e.g., ANTICALINST™; Schlehuber and Skerra, Drug Discov. Today, 10:23-33, 2005), T-cell receptors (e.g., Chlewicki et al., J. Mol. Biol., 346:223-39, 2005), protein A domain (e.g., AFFIBODIES™; Engfeldt et al., ChemBioChem, 6:1043-1050, 2005), Im9 (e.g., Bernath et al., J. Mol. Biol., 345:1015-26, 2005), ankyrin repeat proteins (e.g., DARPins; Amstutz et al., J. Biol. Chem., 280:24715-22, 2005), tetratricopeptide repeat proteins (e.g., Cortajarena et al., Protein Eng. Des. Sel., 17:399-409, 2004), zinc finger domains (e.g., Bianchi et al., J. Mol. Biol., 247:154-60, 1995), pVIII (e.g., Petrenko et al., Protein Eng., 15:943-50, 2002), GCN4 (Sia and Kim, Proc. Natl Acad. Sci. USA, 100:9756-61, 2003), avian pancreatic polypeptide (APP) (e.g., Chin et al., Bioorg. Med. Chem. Lett., 11:1501-5, 2001), WW domains, (e.g., Dalby et al., Protein Sci., 9:2366-76, 2000), SH3 domains (e.g., Hiipakka et al., J. Mol. Biol., 293:1097-106, 1999), SH2 domains (Malabarba et al., Oncogene, 20:5186-5194, 2001), PDZ domains (e.g., TELOBODIES™; Schneider et al., Nat. Biotechnol., 17:170-5, 1999), TEM-1 β-lactamase (e.g., Legendre et al., Protein Sci., 11:1506-18, 2002), green fluorescent protein (GFP) (e.g., Zeytun et al., Nat. Biotechnol., 22:601, 2004), thioredoxin (e.g., peptide aptamers; Lu et al., Biotechnol., 13:366-372, 1995), Staphylococcal nuclease (e.g., Norman, et al., Science, 285:591-5, 1999), PHD fingers (e.g., Kwan et al., Structure, 11:803-13, 2003), chymotrypsin inhibitor 2 (CI2) (e.g., Karlsson et al., Br. J. Cancer, 91:1488-94, 2004), bovine pancreatic trypsin inhibitor (BPTI) (e.g., Roberts, Proc. Natl. Acad. Sci. USA, 89:2429-33, 1992) and many others (see review by Binz et al., Nat. Biotechnol., 23(10):1257-68, 2005 and supplemental materials).

Specific binding reagents also include antibodies. The term “antibody” refers to an immunoglobulin molecule (or combinations thereof) that specifically binds to, or is immunologically reactive with, a particular antigen, and includes polyclonal, monoclonal, genetically engineered and otherwise modified forms of antibodies, including but not limited to chimeric antibodies, humanized antibodies, heteroconjugate antibodies (e.g., bispecific antibodies, diabodies, triabodies, and tetrabodies), single chain Fv antibodies (scFv), polypeptides that contain at least a portion of an immunoglobulin that is sufficient to confer specific antigen binding to the polypeptide, and antigen binding fragments of antibodies. Antibody fragments include proteolytic antibody fragments [such as F(ab′)2 fragments, Fab′ fragments, Fab′-SH fragments, Fab fragments, Fv, and rIgG], recombinant antibody fragments (such as sFv fragments, dsFv fragments, bispecific sFv fragments, bispecific dsFv fragments, diabodies, and triabodies), complementarity determining region (CDR) fragments, camelid antibodies (see, for example, U.S. Pat. Nos. 6,015,695; 6,005,079; 5,874,541; 5,840,526; 5,800,988; and 5,759,808), and antibodies produced by cartilaginous and bony fishes and isolated binding domains thereof (see, for example, International Patent Application No. WO03014161).

A Fab fragment is a monovalent fragment consisting of the VL, VH, CL and CH1 domains; a F(ab′)₂ fragment is a bivalent fragment comprising two Fab fragments linked by a disulfide bridge at the hinge region; an Fd fragment consists of the VH and CHI domains; an Fv fragment consists of the VL and VH domains of a single arm of an antibody; and a dAb fragment consists of a VH domain (see, e.g., Ward et al., Nature 341:544-546, 1989). A single-chain antibody (scFv) is an antibody in which a VL and VH region are paired to form a monovalent molecule via a synthetic linker that enables them to be made as a single protein chain (see, e.g., Bird et al., Science, 242: 423-426, 1988; Huston et al., Proc. Natl. Acad. Sci. USA, 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger et al., Proc. Natl. Acad. Sci. USA, 90:6444-6448, 1993; Poljak et al., Structure, 2:1121-1123, 1994). A chimeric antibody is an antibody that contains one or more regions from one antibody and one or more regions from one or more other antibodies. An antibody may have one or more binding sites. If there is more than one binding site, the binding sites may be identical to one another or may be different. For instance, a naturally occurring immunoglobulin has two identical binding sites, a single-chain antibody or Fab fragment has one binding site, while a “bispecific” or “bifunctional” antibody has two different binding sites.

In some examples, an antibody specifically binds to a target protein (e.g., one of the proteins listed in Table 8, such as WNT5A, TK1, or GAS1) with a binding constant that is at least 10³ M⁻¹ greater, 10⁴ M⁻¹ greater or 10⁵ M⁻¹ greater than a binding constant for other molecules in a sample. In some examples, a specific binding reagent (such as an antibody (e.g., monoclonal antibody) or fragments thereof) has an equilibrium constant (K_{d}) of 1 nM or less. For example, a specific binding agent may bind to a target protein with a binding affinity of at least about 0.1×10⁻⁸ M, at least about 0.3×10⁻⁸M, at least about 0.5×10⁻⁸M, at least about 0.75×10⁻⁸ M, at least about 1.0×10⁻⁸M, at least about 1.3×10⁻⁸ M at least about 1.5×10⁻⁸M, or at least about 2.0×10⁻⁸ M. Kd values can, for example, be determined by competitive ELISA (enzyme-linked immunosorbent assay) or using a surface-plasmon resonance device such as the Biacore T100, which is available from Biacore, Inc., Piscataway, N.J.

Methods of generating antibodies (such as monoclonal or polyclonal antibodies) are well established in the art (for example, see Harlow and Lane, Antibodies: A Laboratory Manual, Cold Spring Harbor Laboratory, New York, 1988). For example peptide fragments of one of the proteins listed in Table 8, such as WNT5A, TK1, or GAS1, (e.g., SEQ ID NO: 2, 4 or 6, respectively) can be conjugated to carrier molecules (or nucleic acids encoding such epitopes or conjugated RDPs) can be injected into non-human mammals (such as mice or rabbits), followed by boost injections, to produce an antibody response. Serum isolated from immunized animals may be isolated for the polyclonal antibodies contained therein, or spleens from immunized animals may be used for the production of hybridomas and monoclonal antibodies. In some examples, antibodies are purified before use.

In one example, monoclonal antibody to one of the proteins listed in Table 8, such as WNT5A, TK1, or GAS1 (e.g., SEQ ID NO: 2, 4 or 6, respectively), can be prepared from murine hybridomas according to the classical method of Kohler and Milstein (Nature, 256:495, 1975) or derivative methods thereof. Briefly, a mouse (such as Balb/c) is repetitively inoculated with a few micrograms of the selected peptide fragment (e.g., epitope of WNT5A, TK1, or GAS1) or carrier conjugate thereof over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Enzymol., 70:419, 1980), and derivative methods thereof. Selected positive clones can be expanded and their monoclonal antibody product harvested for use.

Commercial sources of antibodies include Santa Cruz Biotechnology, Inc. (Santa Cruz, Calif.), Sigma-Aldrich (St. Louis, Mo.), and Abcam (Cambridge, UK). Table 2 shows exemplary commercial sources of antibodies for WNT5A, TK1, and GAS1.

TABLE 2
Exemplary commercial sources of antibodies.

| | Antibody | | |
|---|---|---|---|
| | type | Source | Catalog # |
| WNT5A | Polyclonal | Santa Cruz Biotechnology, Inc. | sc-23698 |
| | Polyclonal | Strategic Diagnostics, Inc. | 2300.00.02 |
| | | (Newark DE) | |
| | Polyclonal | Imgenex (San Diego, CA) | IMG-6075A |
| | Monoclonal | Sigma-Aldrich | W2391 |
| | Monoclonal | Cell Signaling Technology | 2530S |
| | | (Danvers, MA) | |
| TK1 | Monoclonal | Abcam | ab56200 |
| | Monoclonal | Abnova Corporation (Taiwan) | H00007083- |
| | | | M02 |
| | Polyclonal | Abcam | ab56200 |
| | Polyclonal | Abnova Corporation (Taiwan) | H00007083- |
| | | | A01 |
| GAS1 | Polyclonal | Santa Cruz Biotechnology, Inc. | sc-9585; |
| | | | sc-9586 |
| | Polyclonal | R&D Systems (Minneapolis, | AF2636 |
| | | MN) | |
| | Monoclonal | R&D Systems (Minneapolis, | MAB2636 |
| | | MN) | |

Disclosed specific binding agents also include aptamers. In one example, an aptamer is a single-stranded nucleic acid molecule (such as, DNA or RNA) that assumes a specific, sequence-dependent shape and binds to a target protein (e.g., one of the proteins listed in Table 8, such as WNT5A, TK1, or GAS1) with high affinity and specificity. Aptamers generally comprise fewer than 100 nucleotides, fewer than 75 nucleotides, or fewer than 50 nucleotides (such as 10 to 95 nucleotides, 25 to 80 nucleotides, 30 to 75 nucleotides, or 25 to 50 nucleotides). In a specific embodiment, disclosed specific binding reagents are mirror-image aptamers (also called a SPIEGELMER™). Mirror-image aptamers are high-affinity L-enantiomeric nucleic acids (for example, L-ribose or L-2′-deoxyribose units) that display high resistance to enzymatic degradation compared with D-oligonucleotides (such as, aptamers). The target binding properties of aptamers and mirror-image aptamers are designed by an in vitro-selection process starting from a random pool of oligonucleotides, as described for example, in Wlotzka et al., Proc. Natl. Acad. Sci. 99(13):8898-8902, 2002. Methods of generating aptamers are known in the art (see e.g., Fitzwater and Polisky (Methods Enzymol., 267:275-301, 1996; Murphy et al., Nucl. Acids Res. 31:e110, 2003).

In another example, an aptamer is a peptide aptamer that binds to a target protein (e.g., one of the proteins listed in Table 8, such as WNT5A, TK1, or GAS1) with high affinity and specificity. Peptide aptamers include a peptide loop (e.g., which is specific for the target protein) attached at both ends to a protein scaffold. This double structural constraint greatly increases the binding affinity of the peptide aptamer to levels comparable to an antibody's (nanomolar range). The variable loop length is typically 8 to 20 amino acids (e.g., 8 to 12 amino acids), and the scaffold may be any protein which is stable, soluble, small, and non-toxic (e.g., thioredoxin-A, stefin A triple mutant, green fluorescent protein, eglin C, and cellular transcription factor Spl). Peptide aptamer selection can be made using different systems, such as the yeast two-hybrid system (e.g., Gal4 yeast-two-hybrid system) or the LexA interaction trap system.

Specific binding agents optionally can be directly labeled with a detectable moiety. Useful detection agents include fluorescent compounds (including fluorescein, fluorescein isothiocyanate, rhodamine, 5-dimethylamine-1-napthalenesulfonyl chloride, phycoerythrin, lanthanide phosphors, or the cyanine family of dyes (such as Cy-3 or Cy-5) and the like); bioluminescent compounds (such as luciferase, green fluorescent protein (GFP), or yellow fluorescent protein); enzymes that can produce a detectable reaction product (such as horseradish peroxidase, β-galactosidase, luciferase, alkaline phosphatase, or glucose oxidase and the like), or radiolabels (such as ³H, ¹⁴C, ¹⁵N, ³⁵S, ⁹⁰Y, ⁹⁹Tc, ¹¹¹In, ¹²⁵I, or ¹³¹I).

4. Nucleic Acid Probes and Primers

In some examples, the means used to detect one or more (such as at least three) of the genes or gene products listed in Table 8 is a nucleic acid probe or primer. For example, nucleic acid probes or primers specific for the genes listed in Table 8 can be obtained from a commercially available source or prepared using techniques common in the art. Such agents can also be used in the methods provided herein.

Nucleic acid probes and primers are nucleic acid molecules capable of hybridizing with a target nucleic acid molecule (e.g., genomic target nucleic acid molecule). For example, probes specific for a gene listed in Table 8, such as WNT5A, TK1, or GAS1, when hybridized to the target, are capable of being detected either directly or indirectly. Primers specific for a gene listed in Table 8, such as WNT5A, TK1, or GAS1, when hybridized to the target, are capable of amplifying the target gene, and the resulting amplicons capable of being detected either directly or indirectly. Thus probes and primers permit the detection, and in some examples quantification, of a target nucleic acid molecule.

Probes and primers can “hybridize” to a target nucleic acid sequence by forming base pairs with complementary regions of the target nucleic acid molecule (e.g., DNA or RNA, such as cDNA or mRNA), thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share at Least 80% Identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share at Least 50% Identity)

Hybridization: 6×SSC at RT to 55° C. for 16-20 hours

Wash at least twice: 2×-3×SSC at RT to 55° C. for 20-30 minutes each.

Commercial sources of probes and primers include Invitrogen (Santa Cruz, Calif.). Table 3 shows exemplary WNT5A, TK1, and GAS1 primer pairs. Exemplary probes are provided in Table 6 below in the Examples section.

TABLE 3
Exemplary primers.

| | Primer Sets (SEQ ID NO:) |
|---|---|
| WNT5A | 5′-GTGCAATGTCTTCCAAGTTCTTC 3′ (18) |
| | 5′-GGCACAGTTTCTTCTGTCCTTG-3′ (19) |
| | 5′-GGCTGGAAGTGCAATGTCTTCC (20) |
| | 3′-GCCTGTCTTCGCGCCTTCTCC (21) |
| TK1 | 5′- CGC CGG GAA GAC CGT AAT -3′ (22) |
| | 5′- TCA GGA TGG CCC CAA ATG -3′ (23) |
| GAS1 | AATACATTGCTCACCAGGAACC (24) |
| | GTTTAAGGCAGTTTGGAAATGC (25) |

Methods of generating a probe or primer specific for a target nucleic acid (e.g., a gene listed in Table 8, such as WNT5A, TK1, or GAS1) are routine in the art (see e.g., Sambrook et al., (1989) Molecular Cloning, second edition, Cold Spring Harbor Laboratory, Plainview, N.Y.). For example, probes and primers can be generated that are specific for any of SEQ ID NOS: 1, 3 or 5, such as a probe or primer specific for at least 12 to 50 contiguous nucleotides of such sequence (or its complementary strand). Probes and primers are generally at least 12 nucleotides in length, such as at least 15, at least 18, at least 20, at least 25, or at least 30 nucleotides, such as 12 to 100, 12 to 50, 12 to 30 or 15 to 25 nucleotides. Generally, probes include a detectable moiety or “label”. For example, a probe can be coupled directly or indirectly to a “label,” which renders the probe detectable. In some examples, primers include a label that becomes incorporated into the resulting amplicon, thereby permitting detection of the amplicon.

The following examples are provided to illustrate certain particular features and/or embodiments. These examples should not be construed to limit a disclosed invention to the particular features or embodiments described.

### EXAMPLES

### Example 1

### Stringent Controls are Advantageous for Obtaining Reliable Gene Expression Signatures from RNA Isolated from FFPE Tissue Samples

Archiving of scientifically and medically valuable tissue samples (such as those collected from cancer patients) requires long-term stabilization of the otherwise fragile tissues. Formalin fixation and paraffin embedding is one commonly used method for archiving such tissue samples.

RNA isolated from archived FFPE tissue samples is a frequent source for the identification of signatures of genetic abnormalities in cancer (e.g., Gianni et al., J. Clin. Oncol., 23(29):7265-77, 2005; Mina et al., Breast Cancer Res. Treat., 103(2):197-208, 2007). The quality of RNA isolated from such samples will directly affect the outcome of the gene expression analysis.

This Example demonstrates that RNA quality for gene expression analyses may not be inferred from surrogate assays such as qRT-PCR for highly expressed housekeeping genes or by microfluidic separation such as on an Agilent BIOANALYZER™. Instead, more rigorous methods, including those demonstrated in this Example, preferably are used to determine the suitability of RNA samples for such analyses.

### Patient Samples and RNA Isolation

A subset of patient cases (n=28) was selected from the University of Arizona Prostate Cancer Bank for multiplexed mRNA analysis. Individuals with or without prostate cancer recurrence at least five years post-surgery (prostatectomy) were selected for the analysis. Patients presented with either abnormal digital rectal exam (DRE) or elevated serum PSA (>0.4 ng/ml) with normal DRE but subsequent positive sextant biopsy. Cancer recurrence was determined by rising PSA levels. Samples were collected from tissues removed during prostatectomy; then, inked on the surface, fixed overnight in 10% neutral buffered formalin and totally embedded in paraffin blocks using standard methods in the pathology arts. The age of the archived tissue blocks ranged from 6 to 13 years.

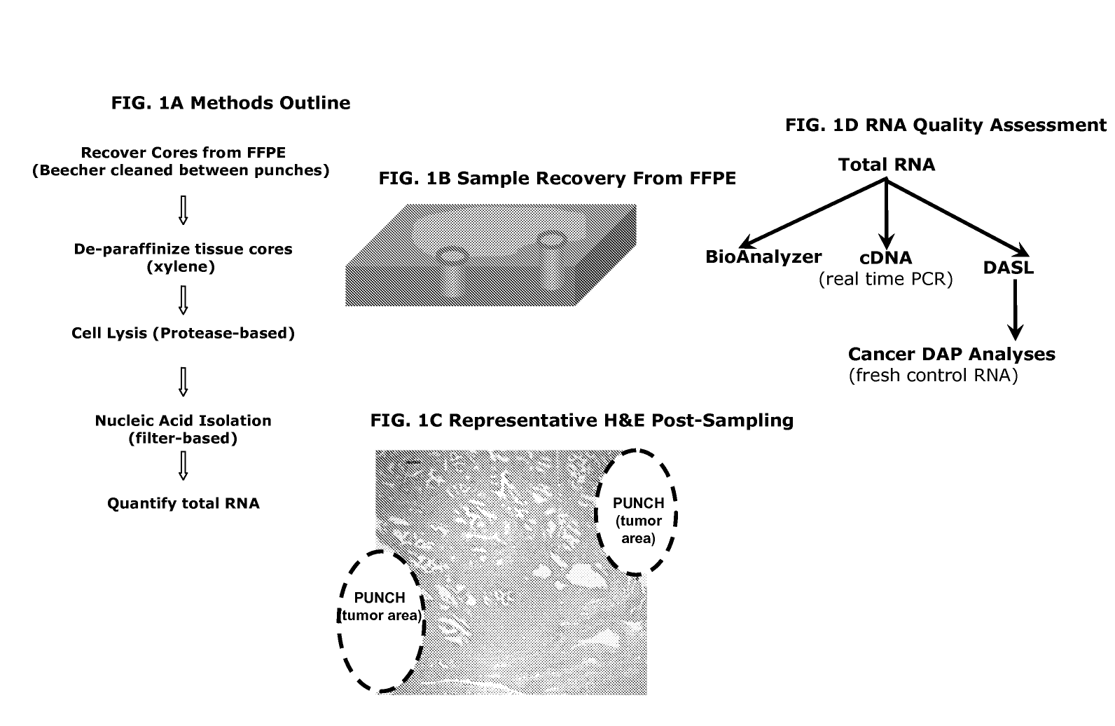

Total RNA was isolated from the test FFPE cores as exemplified in FIGS. 1A-C. Briefly, four micron tissue sections were cut from FFPE tissue blocks from the selected patients. Tissue sections were stained with hematoxylin and eosin (“H&E”) using standard (manual) methods to determine Gleason sum scores, tumor volume, location, and pathologic stage. A Board-certified pathologist reviewed the tissue sections and identified in each section regions of prostate carcinoma. Tissue punches were made in the identified regions and cores collected for RNA isolation. Only men with a minimum 9 year follow-up were included in the study. Recurrence was defined as return of serum PSA greater than 0.3 ng/ml. Fourteen recurrent and fourteen non-recurrent patients were selected for gene expression studies (Table 4).

TABLE 4
Clinical and pathological data
A. Non-recurrent

| | | Presenting | Last | Follow-up | | Gleason |
|---|---|---|---|---|---|---|
| Patient # | Age | PSA | PSA | Time (yrs.) | T-score | Score |
| 4 | 81 | 6.0 | <0.4 | 10.7 | T2c | 3 + 5 = 8/10 |
| 17 | 57 | 4.4 | <0.4 | 7.11 | T2c | 3 + 3 = 6/10 |
| 22 | 66 | 22.0 | <0.4 | 13.10 | T3a | 4 + 5 = 9/10 |
| 23 | 77 | 7.0 | <0.04 | 13.4 | T3a | 4 + 5 = 9/10 |
| 56 | 80 | 3.5 | <0.04 | 12.0 | T2c | 3 + 3 = 6/10 |
| 57 | 85 | 8.0 | <0.4 | 12.2 | T3c | 3 + 3 = 6/10 |
| 58 | 76 | 7.0 | <0.4 | 10.2 | T2c | 3 + 3 = 6/10 |
| 59 | 80 | 14.0 | <0.04 | 10.9 | T4a | 3 + 3 = 6/10 |
| 60 | 77 | 12.8 | <0.4 | 10.8 | T4a | 3 + 4 = 7/10 |
| 61 | 76 | 5.6 | <0.04 | 9.2 | T2c | 3 + 3 = 6/10 |
| 62 | 78 | 11.3 | <0.4 | 8.3 | T3a | 4 + 3 = 7/10 |
| 63 | 64 | 23.0 | <0.04 | 7.8 | T2c | 4 + 4 = 8/10 |
| 64 | 64 | 6.1 | <0.04 | 7.0 | T2c | 3 + 3 = 6/10 |
| 65 | 72 | 8.2 | <0.04 | 7.8 | T3b | 3 + 3 = 6/10 |

B. Recurrent

| | | | | Lag time | | | |
|---|---|---|---|---|---|---|---|
| | | | | from | Follow- | | |
| | | | | surgery to | up | | |
| | | Presenting | Last | recurrence | Time | | Gleason |
| Patient # | Age | PSA | PSA | (yrs.) | (yrs.) | T-score | Score |
| 28 | 79 | 10.1 | 0.23* | 5.4 | 8.4 | T3a | 4 + 3 = 7/10 |
| 29 | 74 | 7.4 | 1.9 | 9.5 | 13.3 | T3a | 3 + 4 = 7/10 |
| 30 | 61 | 48.9 | 1 | 0.5 | 8.2 | T4a | 4 + 3 = 7/10 |
| 31 | 87 | 7.4 | 361.5 | 7.9 | 13.5 | T3a | 4 + 3 = 7/10 |
| 34 | 87 | 5.8 | 826.14 | 5.0 | 7.5 | T3a | 3 + 3 = 6/10 |
| 36 | 79 | 3.6 | 28.53 | 6.10 | 8.6 | T3c | 5 + 4 = 9/10 |
| 38 | 77 | 4.9 | 2.89 | 11.11 | 13.7 | T3a | 4 + 3 = 7/10 |
| 39 | 87 | 12.5 | 2.41 | 12.3 | 13.11 | T3c | 4 + 5 = 9/10 |
| 44 | 77 | 154 | 3893 | 3.5 | 6.2 | T3c | 4 + 4 = 8/10 |
| 46 | 73 | 5.9 | 0.21 | 6.3 | 8.0 | T4a | 4 + 3 = 7/10 |
| 48 | 72 | 14.5 | 1.8 | 2.3 | 7.3 | T3c | 4 + 4 = 8/10 |
| 50 | 73 | 13.4 | 2.68 | 6.4 | 8.2 | T3c | 4 + 3 = 7/10 |
| 51 | 84 | 3.9 | 14 | 0.11 | 8.9 | T4a | 5 + 5 = 10/10 |
| 52 | 71 | 4.5 | 21.6 | 0.7 | 7.4 | T3c | 3 + 3 = 6/10 |

*Patient had an elevated PSA 0.4 in January of 2005, 6 yrs after the surgery.

Representative areas of tumor and adjacent normal were selected by a pathologist using the H&E stained slides from each patient. A Beecher punch was used to manually retrieve cores (1.0 mm diameter, 2-5 mm length) from FFPE blocks into RNase free eppendorf tube for RNA isolation. The coring tool was dipped in xylene and flamed using a Bunsen burner between patient samples to prevent RNA carry over.

The tissue cores from FFPE blocks were deparaffinized in xylene at room temperature for 5 minutes mixing several times and washed twice with ethanol absolute. The tissues then were blotted and dried at 55° C. for 10 minutes. To each tissue pellet 100 μl of tissue lysis buffer containing 16 μl 10% SDS and 40 μl Proteinase K (20 mg/ml) was added and incubated overnight at 55° C. Total RNA was then isolated from the lysed sample using the HIGH PURE™ RNA isolation kit (Roche Applied Science; Indianapolis, Ind., USA). Total RNA was quantified by UV spectroscopy using the NanoDrop-1000 (NanoDrop Technologies Inc., DE) The quantity of RNA was determined with the RIBOGREEN™ assay (Molecular Probes, Eugene, Oreg.). As shown in Table 5, all samples had greater than 400 ng total RNA. A flow diagram of the RNA isolation method is shown in FIG. 1A.

TABLE 5
Quantity of total RNA from RIBOGREEN ™ assay.

| Sample | Plate well | Conc. (ng/μl) | Vol. (μl) | Quantity (ng) |
|---|---|---|---|---|
| TMA #28-R | B01 | 66.50 | 10 | 664.97 |
| TMA #29-R | B02 | 44.95 | 13 | 584.39 |
| TMA #30-R | B03 | 60.65 | 10 | 606.53 |
| TMA #31-R | B04 | 76.24 | 10 | 762.35 |
| TMA #34-R | B05 | 76.82 | 10 | 768.24 |
| TMA #36-R | B06 | 65.76 | 10 | 657.56 |
| TMA #38-R | B07 | 70.13 | 10 | 701.30 |
| TMA #39-R | C01 | 43.45 | 14 | 608.35 |
| TMA #44-R | C02 | 59.88 | 10 | 598.83 |
| TMA #46-R | C03 | 74.49 | 10 | 744.89 |
| TMA #48-R | C04 | 66.75 | 10 | 667.53 |
| TMA #50-R | C05 | 58.67 | 10 | 586.67 |
| TMA #51-R | C06 | 78.43 | 10 | 784.30 |
| TMA #52-R | C07 | 66.38 | 10 | 663.81 |
| TMA #4-NR | D01 | 60.69 | 10 | 606.87 |
| TMA #17-NR | D02 | 67.64 | 10 | 676.37 |
| TMA #22-NR | D03 | 59.42 | 10 | 594.19 |
| TMA #23-NR | D04 | 63.19 | 10 | 631.88 |
| TMA #56-NR | D05 | 63.05 | 10 | 630.52 |
| TMA #57-NR | D06 | 64.84 | 10 | 648.41 |
| TMA #58-NR | D07 | 63.06 | 10 | 630.59 |
| TMA #59-NR | E01 | 64.23 | 10 | 642.29 |
| TMA #60-NR | E02 | 71.06 | 10 | 710.62 |
| TMA #61-NR | E03 | 47.22 | 13 | 613.85 |
| TMA #62-NR | E04 | 58.82 | 10 | 588.18 |
| TMA #63-NR | E05 | 40.40 | 10 | 403.95 |
| TMA #64-NR | E06 | 72.58 | 10 | 725.84 |
| TMA #65-NR | E07 | 67.00 | 10 | 670.04 |

Control RNA samples were freshly isolated from the breast cancer cell line MCF7 or normal breast tissues and quantified using the foregoing methods without the deparaffinization step.

Quantitative Real Time PCR (qRT-PCR)

Quantitative real time PCR (qRT-PCR) was performed on an Applied Biosystems (ABI) 7500 PCR system (SDS v1.4; Applied Biosystems Inc., CA) to qualify samples as potentially useful for DASL® gene expression analysis (Illumina Corporation, CA). The qRT-PCR assay was conducted by measuring the expression of housekeeping gene RPL13a (OMIM Accession No. 113703; GENBANK™ Accession Nos. NM_{—}000977 (GI:15431296) (mRNA variant 1) and NM_{—}033251 (GI:15431294) (mRNA variant 2)) using SYBR® Green RT-PCR Reagents (Applied Biosystems) in conformance with the manufacturers instructions. The forward primer was 5′-GTACGCTGTGAAGGCATCAA-3′ (SEQ ID NO: 7) and the reverse primer was 5′-GTTGGTGTTCATCCGCTTG-3′ (SEQ ID NO: 8), with a resulting amplicon size of 90 bp.

Each reaction contained 25 μL of SYBR Green PCR Master Mix (ABI), 1 μL of cDNA template, and 250 nM each forward and reverse primer in a total reaction volume of 50 μL. All assays were done in triplicate in Micro Amp optical 96-well reaction plates (ABI) closed with Micro Amp optical adhesive covers (ABI). The PCR consisted of an initial enzyme activation step at 95° C. for 10 min, followed by 40 cycles of 95° C. for 15 sec, 60° C. for 1 minute. To access the final product a dissociation curve was generated using a ramp from 60° to 95° C. (ABI).

Relative quantification of the expression level of each transcript in each sample was calculated using the Delta-Delta CT method in the ABI 7500 system software. Normal prostate RNA was used as the calibrator and human Beta Actin (ACTB) gene was used as the endogenous control. Cycle threshold (CT) values were in the range of 19 to 28 and were considered acceptable for analysis by the DASL™ assay. Dissociation curve analysis also yielded a single peak indicating good quality RNA. No significant presence of smaller fragments that would have indicated degradation was observed.

RNA samples were also run on an Agilent BIOANALYZER™ to assess overall RNA quality. RNA quality was determined using the RNA Nano 6000 Series II LabChip (Agilent). All samples pre-qualified by qRT-PCR were judged to be of acceptable quality by the BIOANALYZER™ assessment.

These measures of either the single control gene expression or overall RNAs did not indicate unacceptable levels of degradation in any of the archived samples. Further, no correlation was noted between the age of the blocks and the ability to extract RNA for these analyses.

cDNA Synthesis and DASL Expression Analysis

Total RNA from the 28 original prostate cancer samples and 4 controls were subjected to expression analysis on the Illumina DASL™ BeadChip platform. This cDNA-mediated annealing, selection, extension, and ligation assay (DASL) is designed to generate expression profiles from RNAs including those derived from FFPE tissues (Fan et al., Genome Res. 14:878-85, 2004). The DASL assay was used with the standard Human Cancer Panel from Illumina, which consists of 502 unique cancer genes collected from 10 publicly available cancer gene lists (based on the frequency of appearance of such genes on these lists and the frequency of literature citations of these genes in association with cancer), and with the Universal-16 BeadChip. The assay was performed according to standard Illumina protocols (see, e.g., Illumina BeadStation DASL™ System Manual; Fan et al., Genome Res. 14:878-85, 2004 and Ravo et al., Lab. Invest. 88:430-40, 2008). Briefly, human cancer panel from Illumina comprises a pool of selected probe groups for 502 unique cancer gene mRNAs, each mRNA being targeted in three locations by three separate probes.

For each sample, input quantity for the reaction was normalized to 200 ng (5 ul at 40 ng/ul concentration). This was converted into cDNA using biotinylated random nonamers, oligo-deoxythymidine 18 primers and Illumina-supplied reagents according to manufacturer's instructions. The resulting biotinylated cDNA was annealed to assay oligonucleotides and bound to streptavidin-conjugated paramagnetic particles to select cDNA/oligo complexes. After oligo hybridization, mis-hybridized and non-hybridized oligos were washed away, while bound oligos were extended and ligated to generate templates to be subsequently amplified with shared PCR primers. The fluorescent-labeled complementary strand was hybridized as per standard protocols to Universal DASL 16×1 Bead Chip. Universal-16 Bead Chip platform is composed of 16 individual arrays and for each sample three technical replicates were performed. After hybridization, the arrays were scanned using the Illumina Bead Array Reader 500 system. Intensity data extractions and processing was performed with the Bead Studio Gene Expression Module (GX version 3).

Three sites per transcript were analyzed and data analyses, including for differential gene expression, clustering using rank invariant normalization, and heat maps were all conducted in Bead Studio (Illumina). The heat map used a log (base2) transformation and mean signal subtraction for each gene's unnormalized signal data. Values shown in red on the map are overexpressed relative to the mean; values shown in green are underexpressed relative to the mean; and values shown in black are unchanged relative to the mean.

To validate the DASL assay data (discussed below), qRT-PCR was performed on the test samples based on the manufacturer's instructions with TaqMan gene expression assays (ABI) for the following genes: GAS1, TK1 and WNT5A (assay IDs: Hs00266715_sl, Hs00177406_ml, and Hs00180103_ml). The assay that interrogated the sequence closest to the target sequence in the Illumina platform was chosen (Table 6).

TABLE 6
Illumina and ABI probe details

| Illumina | | Il- | |
|---|---|---|---|
| Accession | Gene | lumina | Illumina probe sequence |
| no. | symbol | start | (SEQ ID NO:) |
| NM_002048.1 | GAS1 | 2051 | GGCGATTGCCTTAGAGGGAACCCC |
| | | | TAAATTGGTTTTGGATAAGTT |
| | | | (9) |
| NM_002048.1 | GAS1 | 1534 | TGGGACAGATAGAAGGGATGGTT |
| | | | GGGGATACTTCCCAAAACTTTTTC |
| | | | (10) |
| NM_003258.1 | TK1 | 1370 | GTGGAGAGGGCAGGGTCCACGCC |
| | | | TCTGCTGTACTTATGAAAT |
| | | | (11) |
| NM_003258.1 | TK1 | 1273 | CTGGTGATGGTTTCCACAGGAACA |
| | | | ACAGCATCTTTCACCAAGAT |
| | | | (12) |
| NM_003258.1 | TK1 | 161 | AGTTGATGAGACGCGTCCGTCGCT |
| | | | TCCAGATTGCTCAGTACAA |
| | | | (13) |
| NM_003392.2 | WNT5A | 2948 | CACTGGGTCCCCTTTGGTTGTAGG |
| | | | ACAGGAAATGAAACATAGGA |
| | | | (14) |
| NM_003392.2 | WNT5A | 804 | CCATATTTTTCTCCTTCGCCCAGGT |
| | | | TGTAATTGAAGCCAATTCTT |
| | | | (15) |
| NM_003392.2 | WNT5A | 597 | GGAGGAGAAGCGCAGTCAATCAA |
| | | | CAGTAAACTTAAGAGACCCCC |
| | | | (16) |
| NM_002048.1 | GAS1 | 1534 | TGGGACAGATAGAAGGGATGGTT |
| | | | GGGGATACTTCCCAAAACTTTTTC |
| | | | (17) |

Note:
Illumina probes were used on DASL platform for expression analysis and ABI probes were used for qRT-PCR on the same set of samples. The ABI Assay ID for GAS1, Hs00266715_s1; for TK1, Hs00177406_ml; and for WNT5A, Hs00180103_ml.

### Results

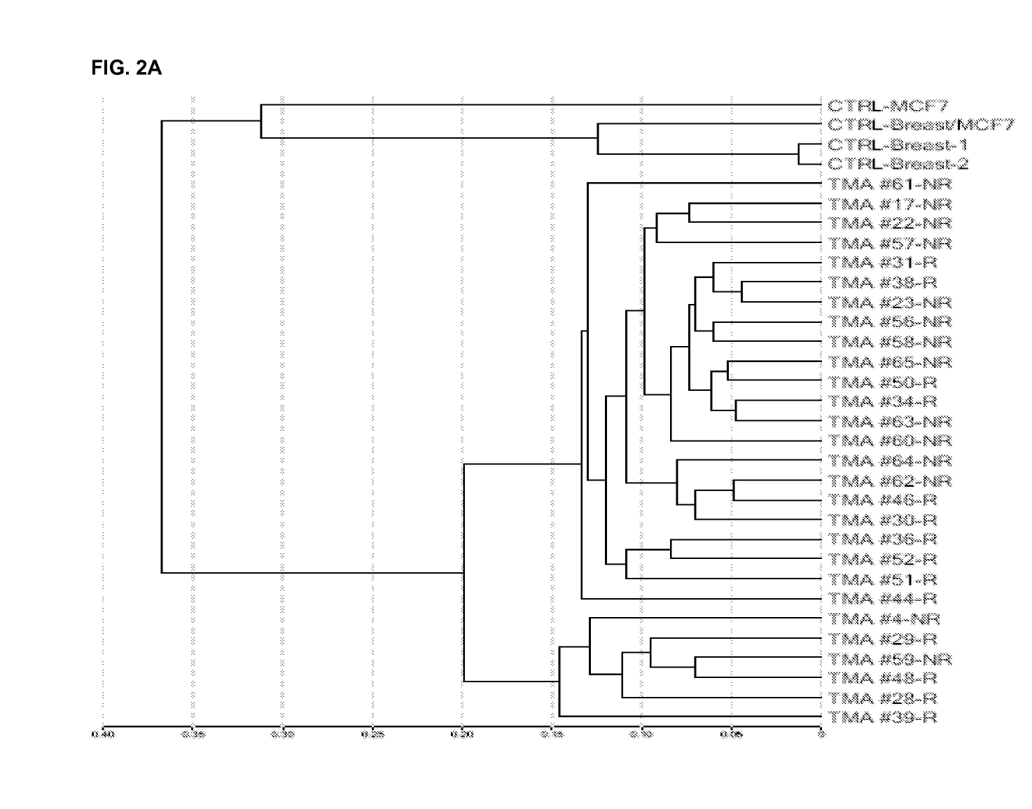

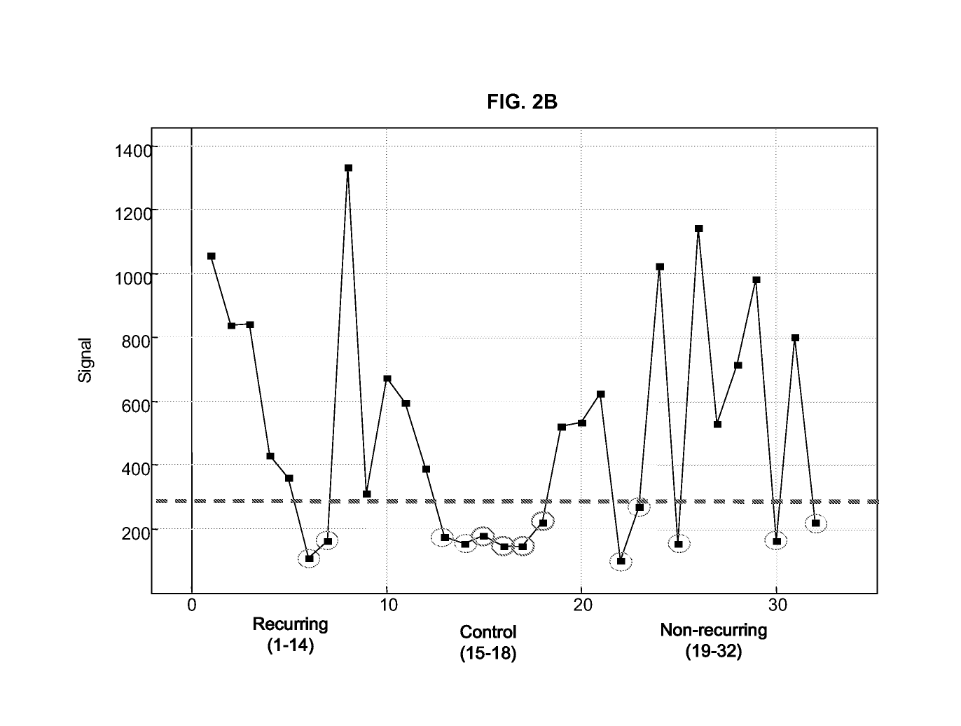

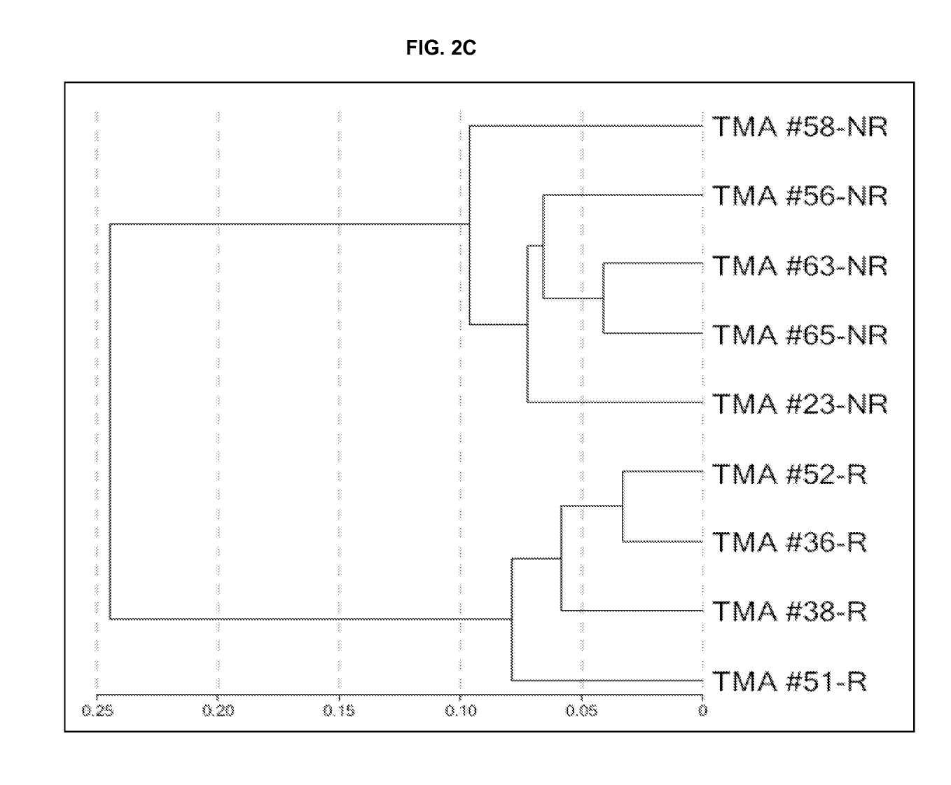

Of the 502 genes analyzed in the Cancer DASL assay pool (DAP), RNA message was detectable for 367 of these genes for all samples. Cluster analysis was performed using rank invariant normalization for all 367 evaluable genes and all samples (24 recurring or non-recurring prostate cancer samples and 4 control breast specimens). The control breast cancer samples (freshly isolated RNA) clustered separately from the prostate cancer samples (see FIG. 2A). In addition, the breast cancer cell line, MCF-7 expressed a profile that distinguished this line from normal cells. These data confirm the expected relationships for breast and prostate cancer (Axelsen et al., Proc. Natl. Acad. Sci. USA 104:13122-7, 2007; Su et al., Cancer Res. 61:7388-93, 2001), as well as for the MCF-7 cell line (Tsai et al., Cancer Res. 67:3845-52, 2007) and normal specimens (Axelsen et al., Proc. Natl. Acad. Sci. USA 104:13122-7, 2007) and demonstrated the suitability of this assay for further analyses of the prostate cancer samples.

Surprisingly, as shown in FIG. 2A, no clear molecular signature for prostate cancer recurrence was determined with unsupervised clustering analyses on all samples for all genes. One explanation for these unexpected results was that the RNA isolated from the prostate samples was of mixed quality causing such samples to cluster together regardless of likelihood of the cancer to recur or not, and that the freshly isolated control RNA was of superior quality causing the control samples to form a distinct cluster.

As shown in FIG. 2B, negative control sample plots showed a significant number of RNA samples with signal >300, which indicates unexpectedly high binding of test samples to irrelevant probe. Thus, the determination of signatures was dependent on the stringency of detection obtained for specific samples. This result may occur if the original RNA samples were more degraded than indicated by qRT-PCR or BIOANALYZER™ assays.

A subset of nine samples having low background reactivity was selected. Samples with “low” background were defined as those with signal comparable to that of the control freshly isolated RNA. Cluster analysis of this sample subset showed a clear distinction between gene expression profiles of recurring and non-recurring prostate cancer samples (FIG. 2C).

The determination of rational gene signatures was dependent on the stringency of detection obtained for specific samples. In particular, samples with a low negative control signals, defined as low binding to irrelevant probes, were found to be most reliable. The outcome of the latter supervised method for gene expression profiling of the present cohort of prostate cancer patients is described in more detail in Example 3.

This Example demonstrates the feasibility of conducting highly multiplexed analyses for mRNA isolated from FFPE tissue. However, RNA quality from FFPE was significantly more degraded than in fresh samples. Thus, the method(s) used to determine the suitability of the RNA samples for these analyses receives additional consideration. For example, RNA quality from FFPE tissue may not be inferred from surrogate assays such as qRT-PCR for highly expressed housekeeping genes or by microfluidic separation such as on the BIOANALYZER™.

Advantageously, a determination of background binding of samples to irrelevant probes (i.e., negative control probes) may serve as a reliable indicator of RNA quality for purposes of gene expression analysis using FFPE samples.

### Example 2

### Prostate Cancer Staging and Recurrence

The clinical parameters for the individuals were subjected to statistical analysis to determine whether there were significant differences between recurrent and non-recurrent sample groups (see Table 4 above).

Clinical parameters, including age, follow-up time, presenting PSA and Gleason score were evaluated with student's t-tests to assess differences in the means between non-recurrent and recurrent subjects. Fisher's exact test was used to detect differences between proportions of T-score. Statistical significance was assessed at p<0.05. These were done using Stata 10 statistical software (StataCorp IC, College Station, Tex.).

Differences among continuous variables (age, follow-up time, presenting PSA and Gleason score) between non-recurrent and recurrent samples were not statistically significant (Table 7). However, the proportion of the subjects having stage T2 was statistically significantly higher in non-recurrent as compared to the recurrent subjects (Table 7). It also was observed that the proportion of subjects having stage T3 was statistically significantly lower in non-recurrent as compared to recurrent subjects (Table 7).

TABLE 7
Comparison of different clinico-pathological parameters between
non-recurrent and recurrent prostate cancer samples

| | Non-recurrent (N = | Recurrent | |
|---|---|---|---|
| Parameters | 15) | (N = 15) | p-value |
| Mean age, yrs (SD) | 73.8 (8.0) | 75.8 (7.0) | 0.48 |
| Mean follow up time, | 121.0 (26.6) | 114.3 (32.2) | 0.55 |
| months (SD) | | | |
| Mean presenting PSA, | 9.9 (6.1) | 20.4 (38.6) | 0.33 |
| ng/ml (SD) | | | |
| Mean Gleason score | 6.7 (1.2) | 7.6 (1.2) | 0.10 |
| (SD) | | | |
| T-score, N (%) | | | |
| T2 | 7 (50.4) | 0 (0) | 0.002* |
| T3 | 5 (35.7) | 12 (80) | 0.02* |
| T4 | 2 (14.3) | 3 (20) | 0.54 |

*statistically significant at p < 0.05

This example shows there were no significant differences between men with indolent prostate disease and men who have progressive disease exhibiting recurrence following prostatectomy among various clinical parameters compared except in the tumor stages and cancer recurrence. Although a higher number of patients having stage T3 were in recurrent group than in non-recurrent group (Table 7), this may not be a strong predictive factor of cancer recurrence since there were a number of cases of patients with high tumor stage in the non-recurrent group as well and two of the non-recurrent cases had obturator lymph node metastasis (T4a) at the time of original surgery (Table 5). This is consistent with previous reports, which showed that selected genes were better predictors of recurrence and independent of tumor grade or stage (Lapointe et al., Proc. Natl. Acad. Sci. USA 101:811-6, 2004).

### Example 3

### Gene Expression Profiling of Patients with Recurring or Non-Recurring Prostate Cancer

This example provides genes that are differentially expressed in patients with recurring and non-recurring prostate cancer. Such information is useful at least to assist in the making of individualized treatment decisions so that patients are not unnecessarily treated and/or are appropriately treated.

As described in Example 1, nine samples, four from patients with recurring prostate cancer (TMA #52-R; TMA #36-R; TMA #38-R; TMA #51-R) and five from patients with non-recurring prostate cancer (TMA #58-NR; TMA #56-NR; TMA #63-NR; TMA #65-NR; TMA #23-NR), were selected for continued analysis based on an acceptably low level of background signal (i.e., low binding to irrelevant (negative-control) probes). Such samples also may be referred to throughout the disclosure at least (or solely) by number (e.g., 52, 36, 38, etc.) in some combination with the designation “NR” (i.e., non-recurring) or “R” (i.e., recurring), as applicable.

Negative controls oligonucleotides targeted 27 random sequences that do not appear in the human genome (Illumina Product Guide 2006/7). The mean signal of these probes defined the system background. The standard deviation of signal on these probes defined the noise. This was a comprehensive measurement of background, and represented the imaging system background as well as any signal resulting from non-specific binding of dye or cross-hybridization. The Bead Studio application used the signals and signal standard deviation of these probes to establish gene expression detection limits.

Using these criteria to select samples for analyses resulted in a 33-gene signature (Table 8) that was identified as significantly (detection p value ≦0.001) differentially expressed between the two groups of prostate cancer and clearly categorized those that recurred or not. The average signal for all 33 genes in each sample is provided in Table 9. The detection p-value shown in Table 9 represents the measure of confidence in signal-to-noise detected for a particular probe set with the test sample. The detection p-value score may be used to filter results to remove particularly noisy samples from subsequent analyses. For the present results, no detection p-value filtering was applied.

TABLE 8
Differentially Expressed Genes That Cluster Prostate Cancer Samples
into Recurring and Non-Recurring Groups

| | | | Functional |
|---|---|---|---|
| ACCESSION | SYMBOL | Full Name | Class |
| NM_033379.2 | CDC2 | cell division cycle 2 | Cell cycle |
| NM_002048.1 | GAS1 | growth arrest-specific 1 | |
| NM_005263.1 | GFI1 | growth factor independent 1 | |
| NM_017579.1 | DMBT1 | deleted in malignant brain | Immune |
| | | tumors 1 | response |
| NM_000758.2 | CSF2 | colony stimulating factor 2 | |
| NM_000575.3 | IL1A | interleukin 1; alpha | |
| NM_012485.1 | HMMR | hyaluronan-mediated | Cell motility |
| | | motility receptor | |
| NM_000059.1 | BRCA2 | breast cancer 2; early onset | Nucleic acid |
| NM_005427.1 | TP73 | tumor protein p73 | metabolism |
| NM_000057.1 | BLM | Bloom syndrome | |
| NM_001951.2 | E2F5 | E2F transcription factor 5 | |
| NM_002315.1 | LMO1 | LIM domain only 1 | |
| NM_000135.1 | FANCA | Fanconi anemia; | DNA repair |
| | | complementation group A | |
| NM_000251.1 | MSH2 | mutS homolog 2; colon | |
| | | cancer; nonpolyposis type 1 | |
| NM_002466.2 | MYBL2 | v-myb myeloblastosis viral | Anti-apoptosis |
| | | oncogene homolog (avian)- | |
| | | like 2 | |
| NM_000499.2 | CYP1A1 | cytochrome P450; family 1 | Energy |
| NM_003258.1 | TK1 | thymidine kinase 1; soluble | pathways |
| NM_000015.1 | NAT2 | N-acetyltransferase 2 | metabolism |
| NM_000301.1 | PLG | plasminogen | Protein |
| NM_002422.2 | MMP3 | matrix metalloproteinase 3 | metabolism |
| NM_003212.1 | TDGF1 | teratocarcinoma-derived | Proliferation |
| | | growth factor 1 | |
| NM_022809.1 | CDC25C | cell division cycle 25C | Proliferation, |
| NM_000697.1 | ALOX12 | arachidonate 12- | cell cycle |
| | | lipoxygenase | |
| NM_003392.2 | WNT5A | wingless-type MMTV | Signal |
| | | integration site family; | transduction |
| | | member | |
| NM_001274.2 | CHEK1 | CHK1 checkpoint homolog | |
| NM_002191.2 | INHA | inhibin; alpha | |
| NM_033163.1 | FGF8 | fibroblast growth factor 8 | |
| NM_004304.3 | ALK | anaplastic lymphoma kinase | |
| NM_004119.1 | FLT3 | fms-related tyrosine | |
| | | kinase 3 | |
| NM_005372.1 | MOS | v-mos Moloney murine | |
| | | sarcoma viral oncogene | |
| | | homolog | |
| NM_198255.1 | TERT | telomerase reverse | Telomere |
| | | transcriptase | maintenance |
| NM_005376.2 | MYCL1 | v-myc myelocytomatosis | Transcriptional |
| | | viral oncogene homolog 1; | control |
| | | lung carcinoma derived | |
| | | (avian) | |
| NM_005378.3 | MYCN | v-myc myelocytomatosis | |
| | | viral related oncogene; | |
| | | neuroblastoma derived | |
| | | (avian) | |

TABLE 9
Average signals and detection p-values for each gene in each subject.

| | #23-NR | | #56-NR | | #58-NR | |
|---|---|---|---|---|---|---|
| | AVG. | Detection | AVG. | Detection | AVG. | Detection |
| SYMBOL | Signal¹ | Pval | Signal | Pval | Signal | Pval |
| CDC25C | −525.013 | 0.319463 | −586.112 | 0.253491 | −1013.14 | 0.857978 |
| E2F5 | 485.8448 | 7.38E−31 | 91.43001 | 2.78E−05 | −578.467 | 0.055334 |
| MMP3 | −556.179 | 0.44855 | −881.574 | 0.78953 | −763.326 | 0.322337 |
| CYP1A1 | −643.807 | 0.795673 | −990.36 | 0.910755 | −984.866 | 0.815371 |
| FGF8 | −658.023 | 0.836776 | −652.643 | 0.369601 | −1002.12 | 0.842241 |
| WNT5A | 2582.846 | 3.68E−38 | 987.5562 | 1.10E−17 | 839.4233 | 3.86E−25 |
| CHEK1 | −552.828 | 0.434131 | −798.143 | 0.651795 | −1011.15 | 0.85523 |
| CSF2 | −675.027 | 0.878339 | −878.868 | 0.785629 | −1036.13 | 0.887307 |
| CDC2 | −523.227 | 0.312536 | −774.043 | 0.606576 | −714.244 | 0.222948 |
| IL1A | −468.85 | 0.139703 | −1016.64 | 0.930028 | −1026.83 | 0.87602 |
| ALK | −460.204 | 0.119809 | −1002.49 | 0.92009 | −1001.56 | 0.841409 |
| MYBL2 | −674.161 | 0.876422 | −864.294 | 0.763905 | −870.519 | 0.577852 |
| MYCL1 | −440.891 | 0.082789 | −762.707 | 0.584752 | −1016.38 | 0.86241 |
| MYCN | −658.025 | 0.836782 | −737.818 | 0.536006 | −1004.37 | 0.845547 |
| TERT | −663.961 | 0.85223 | −565.188 | 0.221383 | −1003.99 | 0.844994 |
| ALOX12 | −607.001 | 0.664542 | −935.253 | 0.858041 | −913.553 | 0.677383 |
| BRCA2 | −600.682 | 0.639073 | −478.795 | 0.115678 | −886.688 | 0.616229 |
| FANCA | −670.519 | 0.868122 | −941.541 | 0.864945 | −879.797 | 0.599988 |
| GAS1 | 2916.224 | 3.68E−38 | 3471.799 | 3.68E−38 | 5019.75 | 3.68E−38 |
| LMO1 | −635.648 | 0.769528 | −976.546 | 0.899158 | −952.288 | 0.757417 |
| PLG | −671.963 | 0.871459 | −962.36 | 0.886143 | −1018.21 | 0.864858 |
| TDGF1 | −653.49 | 0.824296 | −972.721 | 0.895761 | −995.223 | 0.831825 |
| TK1 | 1730.128 | 3.68E−38 | 1854.637 | 9.27E−38 | 1820.839 | 3.68E−38 |
| BLM | −643.155 | 0.79365 | −975.57 | 0.898299 | −640.711 | 0.112496 |
| MSH2 | 500.2026 | 1.19E−31 | −133.243 | 0.001783 | 264.082 | 6.76E−12 |
| NAT2 | −647.867 | 0.807996 | −825.178 | 0.700045 | −811.595 | 0.434444 |
| DMBT1 | −570.899 | 0.512445 | −919.246 | 0.839403 | −873.31 | 0.584541 |
| FLT3 | −634.585 | 0.765988 | −1004.88 | 0.921841 | −561.771 | 0.044789 |
| GFI1 | −107.959 | 2.63E−07 | −786.054 | 0.629336 | −905.625 | 0.659742 |
| MOS | −609.468 | 0.674293 | −940.635 | 0.863964 | −941.334 | 0.735919 |
| TP73 | −534.921 | 0.358995 | −993.963 | 0.91361 | −458.073 | 0.009804 |
| HMMR | −663.535 | 0.851156 | −1013.34 | 0.927805 | −595.107 | 0.067703 |
| INHA | −466.658 | 0.134457 | −710.505 | 0.481912 | −722.85 | 0.239014 |

| | #63-NR | | #65-NR | | #36-R | |
|---|---|---|---|---|---|---|
| | AVG. | Detection | AVG. | Detection | AVG | Detection |
| SYMBOL | Signal | Pval | Signal | Pval | Signal | Pval |
| CDC25C | −768.527 | 0.88187 | −1302.826 | 0.8981501 | −118.8205 | 0.860028 |
| E2F5 | 1018.307 | 1.31E−24 | 591.0235 | 1.44E−19 | 3082.958 | 3.68E−38 |
| MMP3 | −644.132 | 0.65306 | −1063.584 | 0.4907295 | 17.14425 | 0.438058 |
| CYP1A1 | −728.426 | 0.823675 | −1259.845 | 0.8504934 | −98.14562 | 0.813924 |
| FGF8 | −750.999 | 0.858355 | −1291.849 | 0.8871853 | −106.6897 | 0.834012 |
| WNT5A | 1434.778 | 6.38E−38 | 559.4835 | 6.68E−19 | 4704.357 | 3.68E−38 |
| CHEK1 | −669.317 | 0.710108 | −917.7045 | 0.2082635 | 269.3673 | 0.007155 |
| CSF2 | −778.754 | 0.894241 | −1183.298 | 0.7338259 | −58.78362 | 0.703511 |
| CDC2 | −412.953 | 0.140957 | −743.049 | 0.03942797 | 189.1133 | 0.04275 |
| IL1A | −764.832 | 0.877158 | −1307.656 | 0.9027203 | 14.77277 | 0.446571 |
| ALK | −742.052 | 0.845205 | −1232.351 | 0.8132144 | −87.06679 | 0.785734 |
| MYBL2 | −717.407 | 0.804944 | −1216.427 | 0.7892018 | −43.68678 | 0.654408 |
| MYCL1 | −304.016 | 0.038484 | −1296.248 | 0.8916762 | 350.4663 | 0.000719 |
| MYCN | −756.935 | 0.86665 | −1241.413 | 0.8260909 | −129.0219 | 0.879644 |
| TERT | −633.632 | 0.628107 | −986.8716 | 0.3305986 | −41.98421 | 0.648683 |
| ALOX12 | −577.322 | 0.487587 | −1190.827 | 0.7470239 | −31.09992 | 0.611333 |
| BRCA2 | −554.688 | 0.430536 | −1259.833 | 0.8504771 | 6.649379 | 0.475893 |
| FANCA | −598.406 | 0.540984 | −1166.928 | 0.7039722 | −79.94508 | 0.766371 |
| GAS1 | 3654.683 | 3.68E−38 | 2862.735 | 3.68E−38 | 873.1639 | 1.02E−15 |
| LMO1 | −564.817 | 0.45596 | −1242.807 | 0.8280202 | −109.9578 | 0.84131 |
| PLG | −767.799 | 0.880953 | −1266.923 | 0.8592246 | −126.579 | 0.875133 |
| TDGF1 | −740.629 | 0.843041 | −1288.739 | 0.8839309 | −85.86182 | 0.782525 |
| TK1 | 1872.366 | 3.68E−38 | 793.9318 | 3.73E−24 | 4097.804 | 3.68E−38 |
| BLM | −562.102 | 0.449122 | −1038.226 | 0.4362724 | 15.00873 | 0.445723 |
| MSH2 | 1012.275 | 1.94E−24 | 227.6214 | 1.21E−12 | 1621.215 | 3.68E−38 |
| NAT2 | −690.803 | 0.754993 | −1157.015 | 0.6851779 | −101.5803 | 0.822173 |
| DMBT1 | −586.825 | 0.511684 | −1111.517 | 0.5933164 | 78.35807 | 0.238072 |
| FLT3 | −657.807 | 0.684574 | −1201.026 | 0.76434 | −90.44008 | 0.79457 |
| GFI1 | −642.038 | 0.648133 | −1180.96 | 0.7296571 | −45.00525 | 0.658817 |
| MOS | −674.222 | 0.720685 | −943.4676 | 0.2504481 | −70.16383 | 0.738266 |
| TP73 | −511.14 | 0.32569 | −1245.962 | 0.8323363 | 121.1717 | 0.135269 |
| HMMR | −532.904 | 0.376953 | −1305.607 | 0.9008005 | 93.55304 | 0.197472 |
| INHA | −417.733 | 0.147862 | −767.114 | 0.05185061 | 424.8919 | 5.59E−05 |

| | #38-R | | #51-R | | #52-R | |
|---|---|---|---|---|---|---|
| | AVG | Detection | AVG | Detection | AVG | Detection |
| SYMBOL | Signal | Pval | Signal | Pval | Signal | Pval |
| CDC25C | −472.876 | 0.533338 | −778.994 | 0.143984 | −311.978 | 0.656536 |
| E2F5 | 1343.495 | 3.68E−38 | 855.48 | 1.48E−37 | 2635.785 | 3.68E−38 |
| MMP3 | −134.731 | 0.004928 | −1001.78 | 0.702421 | −262.897 | 0.474445 |
| CYP1A1 | −423.162 | 0.379013 | −1066.27 | 0.839587 | −376.231 | 0.844842 |
| FGF8 | −595.585 | 0.853276 | −954.134 | 0.575528 | −398.214 | 0.889483 |
| WNT5A | 4579.362 | 3.68E−38 | 5139.152 | 3.68E−38 | 4576.753 | 3.68E−38 |
| CHEK1 | −424.061 | 0.381711 | −564.051 | 0.004656 | −241.243 | 0.393507 |
| CSF2 | −621.252 | 0.894867 | −1098.76 | 0.889752 | −376.955 | 0.84648 |
| CDC2 | −172.712 | 0.011257 | 450.979 | 3.08E−23 | 28.19581 | 0.002294 |
| IL1A | −562.864 | 0.786039 | −1093.77 | 0.882872 | −412.294 | 0.912735 |
| ALK | −541.95 | 0.734982 | −1086.28 | 0.872004 | −397.51 | 0.888212 |
| MYBL2 | −147.753 | 0.006601 | −153.584 | 1.54E−08 | −312.408 | 0.65804 |
| MYCL1 | −366.175 | 0.224488 | −733.748 | 0.082827 | −219.216 | 0.315671 |
| MYCN | −575.287 | 0.813438 | −1090.11 | 0.877643 | −405.221 | 0.901556 |
| TERT | −80.0878 | 0.0013 | −745.4 | 0.096298 | −391.546 | 0.87704 |
| ALOX12 | −540.907 | 0.732282 | −855.61 | 0.303475 | −272.281 | 0.510058 |
| BRCA2 | −391.753 | 0.289252 | −758.511 | 0.113305 | −308.907 | 0.645723 |
| FANCA | −483.509 | 0.566492 | −688.615 | 0.043706 | −316.572 | 0.672472 |
| GAS1 | 1579.9 | 3.68E−38 | 675.0645 | 1.01E−30 | 300.6003 | 2.87E−08 |
| LMO1 | −579.45 | 0.822112 | −946.927 | 0.555235 | −384.532 | 0.862927 |
| PLG | −607.972 | 0.874556 | −1094.38 | 0.883739 | −395.487 | 0.884509 |
| TDGF1 | −570.011 | 0.802078 | −1061.67 | 0.831428 | −386.276 | 0.866536 |
| TK1 | 2808.751 | 3.68E−38 | 3505.356 | 3.68E−38 | 3160.889 | 3.68E−38 |
| BLM | −326.97 | 0.143201 | −134.644 | 7.05E−09 | −150.685 | 0.1288 |
| MSH2 | 960.398 | 1.86E−29 | 1081.679 | 3.68E−38 | 1714.044 | 3.68E−38 |
| NAT2 | −582.737 | 0.828778 | −1043.41 | 0.796505 | −360.149 | 0.805517 |
| DMBT1 | −445.381 | 0.447099 | −922.43 | 0.485496 | −248.517 | 0.420363 |
| FLT3 | −437.35 | 0.422197 | −886.652 | 0.385013 | −361.448 | 0.808903 |
| GFI1 | −411.049 | 0.343281 | −364.689 | 2.83E−05 | 52.69449 | 0.001078 |
| MOS | −552.305 | 0.761008 | −976.76 | 0.637709 | −331.213 | 0.721097 |
| TP73 | −415.388 | 0.355941 | −1032.32 | 0.77332 | −343.316 | 0.758439 |
| HMMR | −229.021 | 0.033041 | −203.589 | 1.12E−07 | −110.829 | 0.065341 |
| INHA | −155.275 | 0.007782 | −513.475 | 0.001527 | −94.6529 | 0.047919 |

¹In the raw data shown in this table, “AVG. Signal” represents the average of the signals of three unique probes for the indicated gene.

By comparing the average signal (which relates to gene transcript level and, therefore, gene expression level) for a non-recurring sample to the average signal for the same gene in a recurring sample (or vice versa), it is possible to determine the relative expression of the gene between the two samples. For example, in Table 9, the average signal for WNT5A in non-recurring sample 23-NR is 2582.846 and the average signal in recurring sample 36-R is 4704.357. Thus, WNT5A is more highly expressed in the recurring prostate cancer samples.

A similar result can be obtained by comparing WNT5A gene expression in any of the non-recurring samples as compared to any of the recurring samples, or by taking an average of the average signal from all non-recurring samples as compared to an average of the average signal of all of the recurring samples. Analogous comparisons may be performed for each of the genes in Table 9 to determine relative expression (e.g., higher expression in recurring prostate cancer or lower expression in recurring prostate cancer) of such genes. Table 10 shows such averages of the gene expression signals from recurring and non-recurring samples reported in Table 9.

TABLE 10
Averaged Expression Values for Table 9 Genes

| | Averaged AVG. Signal for | Averaged AVG. Signal for All |
|---|---|---|
| SYMBOL | All Recurring Samples | Non-Recurring Samples |
| WNT5A | 4749.906 | 1280.817 |
| TK1 | 3393.2 | 1614.38 |
| E2F5 | 1979.4295 | 321.6277 |
| MSH2 | 1344.334 | 374.1875 |
| GAS1 | 857.182175 | 3585.038 |
| CDC2 | 123.8941275 | −633.503 |
| INHA | −84.6276675 | −616.972 |
| HMMR | −112.471365 | −822.099 |
| BLM | −149.3225675 | −771.953 |
| MYBL2 | −164.35782 | −868.562 |
| GFI1 | −192.012065 | −724.527 |
| CHEK1 | −239.996925 | −789.829 |
| MYCL1 | −242.1681 | −764.049 |
| TERT | −314.7543975 | −770.729 |
| MMP3 | −345.5669125 | −781.759 |
| BRCA2 | −363.1303303 | −756.137 |
| DMBT1 | −384.4924575 | −812.36 |
| FANCA | −392.16032 | −851.438 |
| TP73 | −417.462375 | −748.812 |
| CDC25C | −420.667075 | −839.123 |
| ALOX12 | −424.974455 | −844.791 |
| FLT3 | −443.972495 | −812.013 |
| MOS | −482.6104825 | −821.825 |
| CYP1A1 | −490.952755 | −921.461 |
| LMO1 | −505.216575 | −874.421 |
| IL1A | −513.5379075 | −916.961 |
| FGF8 | −513.655875 | −871.127 |
| NAT2 | −521.969275 | −826.492 |
| TDGF1 | −525.954205 | −930.16 |
| ALK | −528.2017475 | −887.73 |
| CSF2 | −538.93788 | −910.415 |
| MYCN | −549.908825 | −879.712 |
| PLG | −556.10535 | −937.451 |

As with the comparison of the raw data for individual recurring and non-recurring samples, the averaged expression data in Table 10 demonstrates that WNT5A is more highly expressed in the recurring prostate cancer samples. Accordingly, increased WNT5A expression may serve as one exemplary marker of an increased likelihood of prostate cancer recurrence in a human patient. Similarly, the data in Table 10 shows that TK1 is more highly expressed and that GAS1 is less expressed in the recurring prostate cancer samples. Accordingly, increased TK1 and/or decreased GAS1 expression also may serve as exemplary marker(s) of an increased likelihood of prostate cancer recurrence in a human patient.

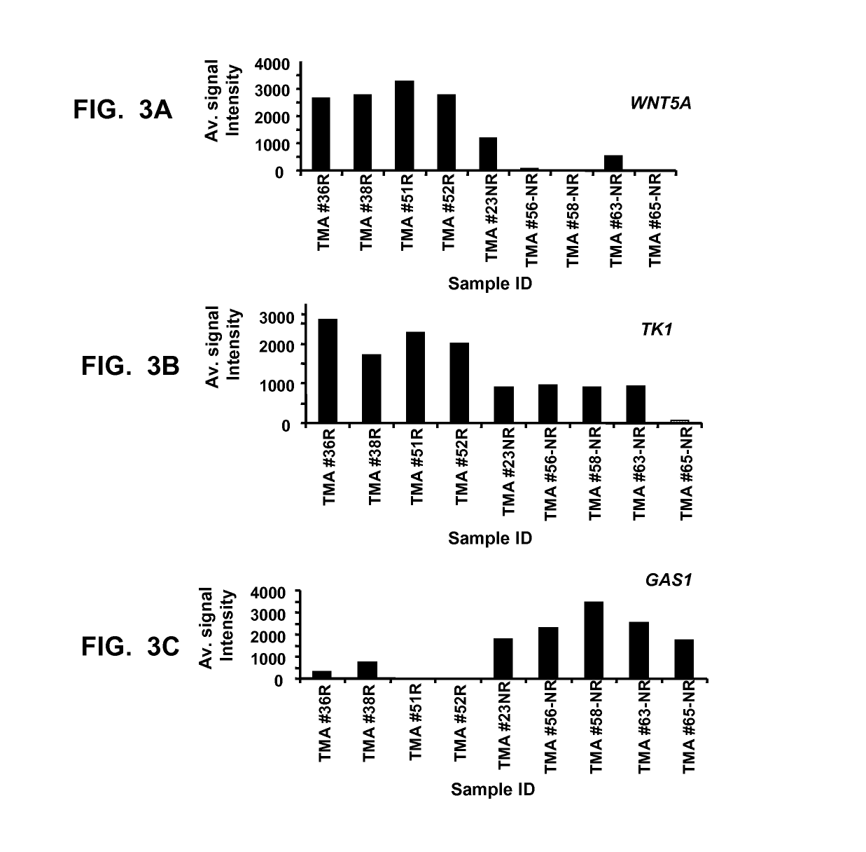

The expression of three exemplary genes, WNT5A, TK1, and GAS1, was more particularly examined. FIGS. 3A-C show the relative expression of such genes in each of the nine samples and clearly demonstrates that recurring and non-recurring prostate cancers further can be distinguished at least by the expression of any one or any combination of these genes. WNT5A and TK1 expression was increased in the recurrent compared to the non-recurrent cases (FIGS. 3A and 3B, respectively). In contrast, GAS1 expression was noticeably increased in the non-recurrent as compared to the recurrent cases (FIG. 3C).

This example is the first to document the over-expression of GAS1 in indolent prostate carcinomas. Using the rat castration model, GAS1 has been shown to be up-regulated in secretory epithelium of the ventral prostate undergoing apoptosis (Bielke et al., Cell. Death Differ. 1997; 4:114-24). Without wishing to be bound to a particular mechanism, increased expression of GAS1 in the non-recurrent cases is believed to result in suppression of proliferation or increase apoptosis.

The subset of 9 samples with differential expression between recurrent and non-recurrent patient groups was subjected to qRT-PCR analysis using ABI TaqMan assay to validate the data obtained on DASL assay. The qRT-PCR assay confirmed the DASL assay expression data at least for WNT5A, TK1, and GAS1.

### Example 4

### Gene Expression Profiling Using GAS1, TK1 and WNT5A

The larger sample set (Table 4, 28 subjects) was used to assess the significance of the differential expression noted for WNT5A, TK1, and GAS1. One outlier sample, from the non-recurrence group (patient # 61), showed high background signal and was also unresponsive across all genes. Thus, 27 samples were analyzed.

The average signal intensities recorded for the 27 individual prostate samples for WNT5A, TK1, and GAS1 were analyzed with the nonparametric Mann-Whitney U-test. The Mann-Whitney U-test, which measures the confidence that two data sets come from separate distributions, indicated that the recurrent and non-recurrent samples for WNT5A and GAS1 showed differences that were statistically significant at the level of p<0.05. The differential expression between non-recurring and recurring for TK1 was significant at p<0.01 (Table 11). There was a striking correlation between the expression of TK1 and recurrence. For TK1, non-recurrent samples are more likely to occur at low expression levels, and recurrent samples at higher expression levels. While GAS1 and WNT5A also show some correlation, more recurrent and non-recurrent samples are found across all expression levels for GAS1 and WNT5A than for TK1. Thus, for this sample set, the distribution of expression levels for non-recurrent and recurrent samples was different for each of the three genes.

TABLE 11
Average signal intensity for 3 gene panel in prostate cancer specimens

| Samples | GAS1 | TK1 | WNT5A |
|---|---|---|---|
| TMA #28-R | 159.1049 | 1814.738 | 1186.885 |
| TMA #29-R | 1482.696 | 1597.475 | 1545.549 |
| TMA #30-R | 243.8143 | 1935.002 | 433.7272 |
| TMA #31-R | 1803.755 | 1676.184 | 1894.676 |
| TMA #34-R | 1692.796 | 1368.922 | −138.5686 |
| TMA #36-R | 309.4907 | 2605.582 | 2667.039 |
| TMA #38-R | 775.5903 | 1721.236 | 2755.156 |
| TMA #39-R | 1972.8 | 579.9047 | 1028.453 |
| TMA #44-R | −217.1194 | 2294.229 | 2554.909 |
| TMA #46-R | 690.5883 | 1399.041 | 3222.52 |
| TMA #48-R | 940.9758 | 1861.906 | 1386.754 |
| TMA #50-R | 774.949 | 1110.019 | −460.8703 |
| TMA #51-R | −119.9042 | 2289.141 | 3255.715 |
| TMA #52-R | −443.0978 | 2008.738 | 2767.253 |
| TMA #4-NR | 692.9727 | 1802.924 | 1712.075 |
| TMA #17-NR | 4039.194 | 1087.417 | 369.8866 |
| TMA #22-NR | 1183.308 | 697.892 | 723.1877 |
| TMA #23-NR | 1806.918 | 902.691 | 1204.131 |
| TMA #56-NR | 2331.08 | 950.781 | 90.11768 |
| TMA #57-NR | 1967.901 | 1969.589 | 628.6682 |
| TMA #58-NR | 3469.622 | 905.8434 | −6.722403 |
| TMA #59-NR | 730.9319 | 1750.327 | 1844.76 |
| TMA #60-NR | 1741.388 | 278.0175 | 461.7899 |
| TMA #62-NR | −722.001 | 834.6077 | 2086.022 |
| TMA #63-NR | 2572.344 | 933.8293 | 536.2645 |
| TMA #64-NR | 66.54094 | 875.1831 | −707.029 |
| TMA #65-NR | 1755.69 | 68.09814 | −132.0587 |

Although the previous tests demonstrated separate recurring and non-recurring distributions for WNT5A, GAS1, and TK1, these distributions do overlap and their ability to reliably predict recurrence is a separate question, which was assessed using logistic regression modeling. Logistic regression analysis was used to develop models that predict the probability of recurrence for individual patients based on their expressed levels of WNT5A, TK1, and GAS1. A commonly used statistic for evaluating the predictions of such models is the area (AUC) under the receiver operating characteristic (ROC) curve constructed from the results. The AUC represents the probability that a randomly selected recurrent patient will have a higher logistic model score than a randomly selected non-recurrent patient. Two cross validation methods were used to estimate the AUC; leave one out cross validation (LOOCV) and 6-fold cross-validation. Both methods partition the samples into a training set (used to calibrate the logistic model parameters) and a test set, from which the AUC is determined. Due to the small number of samples, bootstrap re-sampling was used to improve the AUC estimates, using 100 randomly selected test cases. In the case of leave one out cross validation, each sample was tested against the model trained on all of the other samples, and the results were combined to construct a single ROC curve.

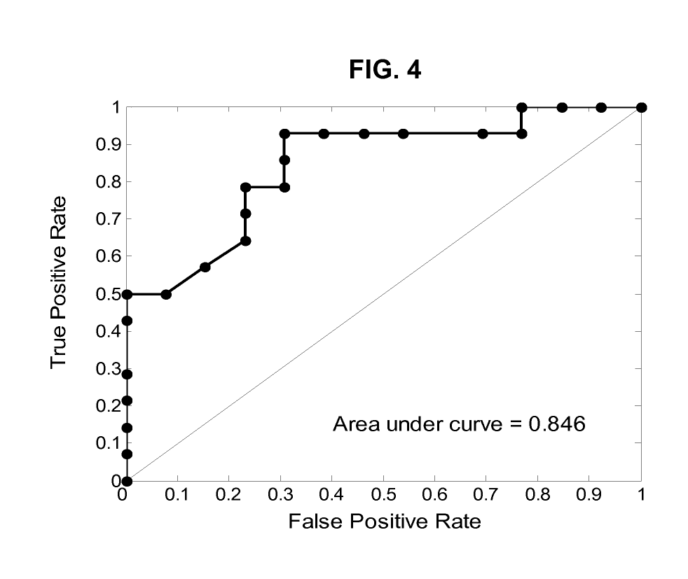

A logistic regression model was fit to the entire set of 27 samples, and an ROC curve was constructed to evaluate how well the model fit the data. An area under the ROC curve (AUC) of 0.846 was achieved for the three gene panel (FIG. 4). This compares favorably with an AUC of 0.758 for the gene panel (SPINK1, PCA3, GOLPH2, and TMPRSS2: ERG) recently identified by Laxman et al. (Cancer Res. 68:645-9, 2008) and 0.508 for the PSA serum test. Thus, in some examples, the disclosed methods have an AUC of at least 0.846.

The ability of the model to predict recurrence for samples not included in the model training set was assessed. Both bootstrapping and leave one out cross validation were employed. An AUC of 0.734 was found using a bootstrapping approach, and an AUC of 0.690 was found using the leave one out cross validation technique. For comparison, Laxman et al. (Cancer Res. 68:645-9, 2008) calculated an AUC of 0.736 for their panel of genes using the leave one out method. Thus, in some examples, the disclosed methods have an AUC of at least 0.690, such as at least 0.734, at least 0.75, at least 0.8, or at least 0.85. For example, if at least GAS1, WNT5A, and TK1 expression levels are determined in a prostate cancer tissue sample, the sensitivity and specificity of determining the prognosis of the subject from whom the sample was obtained is at least 70%, such as at least 75%, at least 80%, at least 85%, at least 90%, at least 92%, at least 95% or at least 98%.

The examples provided herein were performed utilizing highly multiplexed biomarker assays based on mRNA recovered from widely available archival FFPE tissues with the goal to identify low complexity molecular signatures to predict prostate cancer recurrence, which can be utilized in routine clinical pathology practice. The results provided herein provide a number of genes (Table 8) the expression of which (either individually or in any combination) can be used to distinguish between a prostate cancer that will or will not recur, e.g., after prostatectomy surgery. Thus, any one or more (such as any two, three, four, five or six) or any combination of the genes in Table 8 can be used (at least) to determine the likelihood of prostate cancer recurrence in a patient. One exemplary gene signature identified by this method is characterized by over-expression of WNT5A and TK1 and down-regulation of GAS1. This novel three gene signature distinguished recurrent and non-recurrent prostate cancers in surgical specimens removed at least five years prior to follow-up. The results herein further show that the ability of these three genes to predict the likelihood of the prostate cancer recurrence is significantly better than the PSA serum test.

### Example 5

### In Situ Hybridization to Detect Expression

This example provides exemplary methods that can be used to detect gene expression using in situ hybridization, such as FISH or CISH. Although particular materials and methods are provided, one skilled in the art will appreciate that variations can be made.

Prostate cancer tissue samples, such as FFPE samples, are mounted onto a microscope slide, under conditions that permit detection of nucleic acid molecules present in the sample. For example, cDNA or mRNA in the sample can be detected. The slide is incubated with nucleic acid probes that are of sufficient complementarity to hybridize to cDNA or mRNA in the sample under very high or high stringency conditions. Probes can be RNA or DNA. Separate probes that are specific for GAS1, TK1, and WNT5A nucleic acid sequences (e.g., human sequences) are incubated with the sample simultaneously or sequentially, or incubated with serial sections of the sample. For example, each probe can include a different fluorophore or chromogen to permit differentiation between the three probes. After contacting the probe with the sample under conditions that permit hybridization of the probe to its gene target, unhybridized probe is removed (e.g., washed away), and the remaining signal detected, for example using microscopy. In some examples, the signal is quantified.

In some examples, additional probes are used, for example to detect expression of one or more other genes listed in Table 8, or one or more housekeeping genes (e.g., β-actin). In some examples, expression of GAS1, TK1, and WNT5A is also detected (using the same probes) in a control sample, such as a breast cancer cell, a prostate cancer cell from a subject who has not had a recurring prostate cancer, a prostate cancer cell from a subject who had a recurring prostate cancer, or a normal (non-cancer) cell.

The resulting hybridization signals for GAS1, TK1, and WNT5A are compared to a control, such as a value representing GAS1, TK1, and WNT5A expression in a non-recurring cancer or in a recurring cancer. If increased expression of TK1 and WNT5A, and decreased expression of GAS1, relative to a value representing GAS1, TK1, and WNT5A expression in a non-recurring cancer, this indicates that the subject has a poor prognosis (e.g., less than a 1 or 2 year survival) as the cancer is likely to recur. Similarly, if GAS1, TK1, and WNT5A expression is similar relative to a value representing GAS1, TK1, and WNT5A expression in a recurring cancer, this indicates that the subject has a poor prognosis as the cancer is likely to recur. If GAS1, TK1, and WNT5A expression is similar (e.g., no more than a 2-fold difference) relative to a value representing GAS1, TK1, and WNT5A expression in a non-recurring cancer, this indicates that the subject has a good prognosis as the cancer is not likely to recur.

### Example 6

### Nucleic Acid Amplification to Detect Expression

This example provides exemplary methods that can be used to detect gene expression using nucleic acid amplification methods, such as PCR. Amplification of target nucleic acid molecules in a sample can permit detection of the resulting amplicons, and thus detection of expression of the target nucleic acid molecules. Although particular materials and methods are provided, one skilled in the art will appreciate that variations can be made.

RNA is extracted from a prostate cancer tissue sample, such as FFPE samples or fresh tissue samples (e.g., surgical specimens). Methods of extracting RNA are routine in the art, and exemplary methods are provided elsewhere in the disclosure. For example RNA can be extracted using a commercially available kit. The resulting RNA can be analyzed as described in Example 1 to determine if it is of an appropriate quality and quantity.

The resulting RNA can be used to generate DNA, for example using RT-PCR, such as qRT-PCR. Methods of performing PCT are routine in the art. For example, the RNA is incubated with a pair of oligonucleotide primers specific for the target gene (e.g., GAS1, WNT5A, and TK1). Such primers are of sufficient complementarity to hybridize to the RNA under very high or high stringency conditions. Primer pairs specific for GAS1, TK1, and WNT5A nucleic acid sequences (e.g., human sequences) can be incubated with separate RNA samples (e.g., three separate PCR reactions are performed), or a plurality of primer pairs can be incubated with a single sample (for example if the primer pairs are differentially labeled to permit a discrimination between the amplicons generated from each primer pair). For example, each primer pair can include a different fluorophore to permit differentiation between the amplicons. Amplicons can be detected in real time, or can be detected following the amplification reaction. Amplicons are usually detected by detecting a label associated with the amplicon, for example using spectroscopy. In some examples, the amplicon signal is quantified.

In some examples, additional primer pairs are used, for example to detect expression of one or more other genes listed in Table 8, or one or more housekeeping genes (e.g., β-actin). In some examples, expression of GAS1, TK1, and WNT5A is also detected (using the same probes) in a control sample, such as a breast cancer cell, a prostate cancer cell from a subject who has not had a recurring prostate cancer, a prostate cancer cell from a subject who had a recurring prostate cancer, or a normal (non-cancer) cell.

The resulting amplicon signals for GAS1, TK1, and WNT5A are compared to a control, such as a value representing GAS1, TK1, and WNT5A expression in a non-recurring cancer or in a recurring cancer. If increased expression of TK1 and WNT5A, and decreased expression of GAS1, relative to a value representing GAS1, TK1, and WNT5A expression in a non-recurring cancer, this indicates that the subject has a poor prognosis (e.g., less than a 1 or 2 year survival) as the cancer is likely to recur. Similarly, if GAS1, TK1, and WNT5A expression is similar relative to a value representing GAS1, TK1, and WNT5A expression in a recurring cancer, this indicates that the subject has a poor prognosis as the cancer is likely to recur. If GAS1, TK1, and WNT5A expression is similar (e.g., no more than a 2-fold difference) relative to a value representing GAS1, TK1, and WNT5A expression in a non-recurring cancer, this indicates that the subject has a good prognosis as the cancer is not likely to recur.

1. A method of characterizing a prostate cancer tissue, comprising determining in a prostate tissue sample from a subject having prostate cancer the expression level of one or more prognostic genes, which comprise WNT5A, TK1, or GAS1 or any combination thereof, as compared to a control standard or the expression of the prognostic genes in a control sample; wherein differential expression of WNT5A, TK1, or GAS1 or any combination thereof in the prostate tissue sample as compared to the control standard or the expression of the prognostic genes in a control sample characterizes the prostate cancer tissue.

2. The method of claim 1, wherein characterizing a prostate cancer tissue comprises predicting the likelihood of disease recurrence after prostatectomy or predicting the likelihood of prostate cancer progression.

3. The method of claim 2 wherein the one or more prognostic genes further comprise any one or more other genes or combination of other genes listed in Table 8; wherein increased expression of the other genes indicates a lower likelihood of recurrence of prostate cancer in the subject, and wherein the other genes are not WNT5A, TK1, or GAS1.

4. The method of claim 1, wherein determining the expression level comprises measuring the level of an expression product of each of the one or more prognostic genes.

5. The method of claim 1, wherein the expression product is an mRNA or a protein.

6. The method of claim 1, wherein determining the expression level comprises detecting alteration(s) in the genomic sequence(s) of the one or more prognostic genes.

7. The method of claim 6, wherein the alteration in the genomic sequence is amplification of at least one WNT5A or TK1 allele or deletion of at least one GAS1 allele.

8. The method of claim 1, wherein the one or more prognostic genes consist of WNT5A, TK1, or GAS1, or any combination thereof.

9. The method of claim 1, wherein the prognostic gene comprises GAS1.

10. The method of claim 5, wherein the one or more prognostic genes consist of WNT5A, TK1, and GAS1.

11. The method of claim 1, wherein the prostate tissue sample is a fixed, wax-embedded prostate tissue sample.

12. The method of claim 2, wherein the prostate tissue sample is collected after prostate cancer diagnosis and prior to prostatectomy in the subject.

13. The method of claim 2, wherein the prostate tissue sample is collected from tissue removed during the prostatectomy.

14. The method of claim 2, wherein disease recurrence occurs within 5 years of the prostatectomy.

15. A kit for predicting the likelihood of prostate cancer progression, comprising means for detecting in a biological sample WNT5A genomic sequence, WNT5A transcript or WNT5A protein, means for detecting in a biological sample TK1 genomic sequence, TK1 transcript or TK1 protein, or means for detecting in a biological sample GAS1 genomic sequence, GAS1 transcript or GAS1 protein, or any combination of any of the foregoing.

16. The kit of claim 15, comprising means for detecting in a biological sample WNT5A transcript or protein, means for detecting in a biological sample TK1 transcript or protein, and means for detecting in a biological sample GAS1 transcript or protein.

17. The kit of claim 15, comprising a nucleic acid probe specific for WNT5A transcript, a nucleic acid probe specific for TK1 transcript, and a nucleic acid probe specific for GAS1 transcript.

18. The kit of claim 15, comprising a pair of primers for specific amplification of WNT5A transcript, a pair of primers for specific amplification of TK1 transcript, and a pair of primers for specific amplification of GAS1 transcript.

19. The kit of claim 15, comprising an antibody specific for WNT5A protein, an antibody specific for specific for TK1 protein, and an antibody specific for a GAS1 protein.

20. The kit of claim 15, comprising at least two detection means selected from the group consisting of:
a nucleic acid probe specific for WNT5A transcript, a nucleic acid probe specific for TK1 transcript, a nucleic acid probe specific for GAS1 transcript, a pair of primers for specific amplification of WNT5A transcript, a pair of primers for specific amplification of TK1 transcript, a pair of primers for specific amplification of GAS1 transcript, an antibody specific for WNT5A protein, an antibody specific for specific for TK1 protein, and an antibody specific for a GAS1 protein.

21. The kit of claim 20 comprising at least three detection means selected from the group consisting of:
a nucleic acid probe specific for WNT5A transcript, a nucleic acid probe specific for TK1 transcript, a nucleic acid probe specific for GAS1 transcript, a pair of primers for specific amplification of WNT5A transcript, a pair of primers for specific amplification of TK1 transcript, a pair of primers for specific amplification of GAS1 transcript, an antibody specific for WNT5A protein, an antibody specific for specific for TK1 protein, and an antibody specific for a GAS1 protein.

22. The kit of claim 20, further comprising a detection means selected from the group consisting of:
a nucleic acid probe specific for a housekeeping gene transcript, a pair of primers for specific amplification of a housekeeping gene transcript, and an antibody specific for a housekeeping protein.

23. An array consisting of nucleic acid probes specific for a transcript of from each of the following genes: CDC25C, E2F5, MMP3, CYP1A1, FGF8, WNT5A, CHEK1, CSF2, CDC2, IL1A, ALK, MYBL2, MYCL1, MYCN, TERT, ALOX12, BRCA2, FANCA, GAS1, LMO1, PLG, TDGF1, TK1, BLM, MSH2, NAT2, DMBT1, FLT3, GFI1, MOS, TP73, HMMR, and INHA.

24. An array consisting of nucleic acid probes specific for a WNT5A transcript, a TK1 transcript, and a GAS1 transcript.

25. An array consisting of nucleic acid probes specific for a WNT5A transcript, a TK1 transcript, a GAS1 transcript, and a housekeeping transcript.
